(19) United States
(12) Patent Application Publication
(10) Pub. No.: US 2014/0030261 A1
(43) Pub. Date: Jan. 30, 2014

(54) METHOD FOR PROGNOSTICATING THE CLINICAL RESPONSE OF A PATIENT TO B-LYMPHOCYTE INHIBITING OR DEPLETING THERAPY IN INTERFERON-DRIVEN DISEASES SUCH AS SLE

(71) Applicant: Cornelis Lammert Verweij, Abcoude (NL)

(72) Inventor: Cornelis Lammert Verweij, Abcoude (NL)

(73) Assignee: Vereniging Voor Christelijk Hoger Onderwijs, Wetenschappelijk Onderzoek En Patientenzorg, Amsterdam (NL)

(21) Appl. No.: 13/990,772

(22) PCT Filed: Nov. 30, 2011
(86) PCT No.: PCT/NL2011/050819

§ 371 Date: Oct. 9, 2013

Related U.S. Application Data
(60) Provisional application No. 61/458,785, filed on Nov. 30, 2010.

(51) Int. Cl. C12Q 1/68

(52) U.S. Cl. CPC C12Q 1/6881
USPC 424/133.1; 506/9; 435/6.12

(57) ABSTRACT

Described are methods for predicting a clinical response to B-lymphocyte inhibiting or depleting therapies (BCIDT) using expression levels of genes of the Type I INF pathway. In another aspect, the disclosure relates to a method for evaluating a pharmacological effect of a treatment with B-lymphocyte inhibiting or depleting therapy. More in particular, it relates to a method for prognosticating the clinical response of a patient to treatment with a soluble BCID or TCID agent, the method comprising the steps of obtaining at least two samples from the patient wherein a first sample has not been exposed to a soluble BCID or TCID agent and wherein at least a second sample has been exposed to a soluble BCID or TCID agent, determining the level of an IFN (preferably type I) response in the at least two samples, comparing the level of the IFN (preferably type I) response in the first sample with the level of the IFN (e.g., type I) response in the at least second sample and prognosticating the clinical response from the comparison.

### CROSS-REFERENCE TO RELATED APPLICATIONS

This application is a national phase entry under 35 U.S.C. §371 of International Patent Application PCT/NL2011/050819, filed Nov. 30, 2011, designating the United States of America and published in English as International Patent Publication WO 2012/074396 A1 on Jun. 7, 2012, which claims the benefit under Article 8 of the Patent Cooperation Treaty and under 35 U.S.C. §119(e) to U.S. Provisional Patent Application Ser. No. 61/458,785, filed Nov. 30, 2010.

### TECHNICAL FIELD

The disclosure relates to biotechnology and methods for predicting a clinical response to B-lymphocyte inhibiting or depleting therapies (BCIDT) using expression levels of genes of the INF pathway, preferably the type I IFN pathway. In another aspect, it relates to a method for evaluating a pharmacological effect of a treatment with B-lymphocyte inhibiting or depleting therapy. More in particular, it relates to a method for prognosticating the clinical response of a patient to treatment with a soluble BCID or TCID agent, the method comprising the steps of obtaining: i, one sample not exposed to a soluble BCID or TCID before the start of treatment with a soluble BCID or TCID, or ii, at least two samples from the patient, wherein a first sample has not been exposed to a soluble BCID or TCID agent and wherein at least a second sample has been exposed to a soluble BCID or TCID agent.

The prediction is based on determining the level of an interferon (IFN) response signature in the single sample and prognosticating the clinical response from the measurement. Alternatively, the prediction is based on determining the level of an IFN response in the at least two samples, comparing the level of the IFN response in the first sample with the level of the IFN response in the at least second sample and prognosticating the clinical response from the comparison. This prediction rule can also be applied to prognosticate the response to IFNs.

More specifically, the disclosure relates to a method for prognosticating the clinical response of a patient with a disease such as SLE, whereby IFNs (preferably, type I) are related with disease pathogenesis and/or disease activity, who are treated with a soluble BCID or TCID agent, the method comprising the steps of:
1. Obtaining a sample that has not been exposed to soluble BCIDT or TCIDT before the start of therapy and prognosticate the clinical response by comparing the level of IFN response gene expression to a cut-off point;
2. a. Obtaining at least two samples from the patient, wherein a first sample has not been exposed to a soluble BCID or TCID agent and wherein at least a second sample has been exposed to a soluble BCID or TCID agent,
   b. Obtaining at least two samples from the patient, wherein a first sample has not been exposed to an IFN (preferably, type I) or IFN-inducing agent (preferably, type I) such as RNA or DNA and wherein at least a second sample has been exposed to an IFN (preferably, type I) or I IFN-inducing agent (preferably, type I) such as RNA and DNA,
   b. Determining the level of an IFN (preferably, type I) response in the at least two samples,
   c. Comparing the level of the IFN (preferably, type I) response in the first sample with the level of the IFNe (preferably, type I) response in the at least second sample, and
   d. Prognosticating the clinical response from the comparison.

### DISCLOSURE

Described is a method is for prognosticating the clinical response of a patient with a disease, wherein IFN contributes to disease activity and/or severity like SLE to treatment with a soluble BCID or TCID agent, the method comprising the steps of
a. Obtaining at least two samples from the patient, wherein a first sample has not been exposed to a soluble BCID or TCID agent and wherein at least a second sample has been exposed to a soluble BCID or TCID agent,
b. Determining the level of an IFN-I type response in the at least two samples,
c. Comparing the level of the IFN-I type response in the first sample with the level of the IFN-I type response in the at least second sample, and
d. Prognosticating the clinical response from the comparison.

Provided is a method for prognosticating the clinical response of a patient with a disease, wherein IFN contributes to disease activity and/or severity like SLE to treatment with a soluble BCID or TCID agent, the method comprising the steps of:
a. Obtaining at least two samples from the patient, wherein a first sample has not been exposed to a soluble BCID or TCID agent, IFN type I bioactivity or IFN type I-inducing agents and wherein at least a second sample has been exposed to a type I IFN or a type I-inducing agent such as dsDNA or dsRNA,
b. Determining the level of an IFN-I type response in the at least two samples,
c. Comparing the level of the IFN-I type response in the first sample with the level of the IFN-I type response in the at least second sample,
d. Prognosticating the clinical response from the comparison.

Provided is a method for prognosticating the clinical response of a patient with a disease, wherein IFN contributes to disease activity and/or severity like SLE to treatment with a soluble BCID or TCID agent prior to the start of therapy in a single sample taken prior to the start of therapy, the method comprising the steps of:
a. Obtaining one sample not exposed to a soluble BCID or TCID before the start of treatment with a soluble BCID or TCID,
b. Determine the level of an IFN response and/or protein metabolism signature (for the latter, the increased expression at baseline is associated with a good clinical response),
c. Comparing the level of the IFN-I type response gene signature (FIGS. 2 and 3, Tables 1 and 2) in the single sample with predetermined cut-off values, and
d. Prognosticating the clinical response from the comparison prior to the start of therapy.

Provided is a method for prognosticating the clinical response of a patient with a disease, wherein IFN contributes to disease activity and/or severity like SLE to treatment with a type I IFN or type I IFN-inducing agent, the method comprising the steps of:
a. Obtaining at least two samples from the patient, wherein a first sample has not been exposed to a soluble BCID or TCID agent and wherein at least a second sample has been exposed to a soluble BCID or TCID agent,
b. Determining the level of an IFN-I type response in the at least two samples,
c. Comparing the level of the IFN-I type response in the first sample with the level of the IFN-I type response in the at least second sample, and
d. Prognosticating the clinical response from the comparison.

Provided is a method for prognosticating the clinical response of a patient with a disease, wherein IFN contributes to disease activity and/or severity like SLE to treatment with a type I IFN or a type I IFN-inducing agent prior to the start of therapy in a single sample taken prior to the start of therapy, the method comprising the steps of:
a. Obtaining one sample not exposed to a soluble BCID or TCID before the start of treatment with a soluble BCID or TCID,
b. Determine the level of an IFN type response and/or protein metabolism signature (for the latter, the increased expression at baseline is associated with a good clinical response),
c. Comparing the level of the IFN-I type response and/or protein metabolism gene signature (Tables 1 and 2, FIGS. 2 and 3) in the single sample with predetermined cut-off values, and
d. Prognosticating the clinical response from the comparison prior to the start of therapy.

The IFN-I type response level may be determined by determining the expression level of BAFF and DARC genes supplemented with at least one gene selected from the group consisting of genes from Tables 1A, 1B, 1C, 1D and 2. The IFN-I type response level may be determined by determining the level of an expression product of at least one gene selected from the group consisting of OAS1 and MX2.

The IFN-I type response level may be determined by determining the level of an expression product of at least one gene selected from the group consisting of Mx1, ISG15, OAS1, LGALS3BP, RSAD2, IFI44L, IFI44, MX2, OAS2, DARC, BAFF, HERC5, Ly6E, IFI27, RAP1GAP, EPSTI1 and/or SERPING1.

The IFN-I type response level may be determined by determining the level of an expression product of at least one gene selected from the group consisting of RSAD2 and IFI44L.

The IFN-I type response level may be determined by determining the level of an expression product of at least one gene selected from the group consisting of Mx1, ISG15, OAS2 and SERPING1.

The IFN-I type response level may be determined by determining the level of an expression product of a gene selected from the group consisting of genes listed in Tables 1A, 1B, 1C, 1D, and 2, BAFF and DARC.

Preferably, the samples comprise cells and serum/plasma. Preferably, the sample comprises cells and serum/plasma from the patient before the start of the therapy to predict the response to the soluble BCID or TCID agent. Preferably, the at least a second sample is obtained from an individual between one and eight months after the first exposure of the individual to the soluble BCID or TCID agent. Preferably, the at least a second sample also taken at baseline (simultaneously with sample one prior to the start of therapy) has been exposed in vitro to the soluble BCID or TCID agent. Preferably, the at least a second sample also taken at baseline (simultaneously with sample one prior to the start of therapy) has been exposed in vitro to type I IFN or a type I-inducing agent such as dsDNA or dsRNA. Preferably, the at least a second sample has been obtained from a patient that has been exposed to a BCID or TCID agent.

In a further embodiment, a method for treatment of an individual suffering from or at risk of suffering from a B- and/or T-cell-related disease with a soluble BCID or TCID agent is provided, comprising the steps of:
a. determining a prognosis for a clinical response to a treatment with the soluble BCID or TCID agent according to any one of the embodiments of the invention,
b. treating the individual with the soluble BCID or TCID agent, if the individual has been prognosticated as a good responder.

In a further embodiment, a method is provided for prognosticating the therapeutic response to, and/or pharmacodynamic effects of soluble BCIDT and/or TCIDT using genetic markers in the gene for IRF5 that determine the IFN response activity in blood (rs2004640 rs10954213, rs4728142 and a 30 by indel polymorphism), serum proteins such as BAFF known to correlate with IFN response activity, and phenotypic markers on blood cells such as SiglecI and CD64 on peripheral blood cells such as monocytes.

The disclosure is based, in part, on the finding that the response to a BCID or TCID, such as rituximab, in a patient afflicted with a disease, such as SLE, can be predicted based on determining an interferon response. Accordingly, in one aspect, the disclosure provides a method for prognosticating the clinical response to a B-lymphocyte inhibiting or depleting agent (BCID) or a T-cell inhibiting or depleting agent (TCID) in a patient afflicted with a disease, wherein IFN contributes to the disease, disease activity and/or severity, the method comprising:
providing a sample from the patient, preferably, wherein the patient has not previously been exposed to a BCID or TCID agent, preferably, wherein the patient has also not been exposed to an IFN or IFN-inducing agent,
determining the level of an IFN response in the sample, and
prognosticating the clinical response from the IFN response,

wherein a low level of IFN response indicates the likelihood of a poor clinical response to the BCID or TCID.

It is preferred that the patient has not previously been exposed to a BCID or TCID agent, or rather, that the sample represents a “baseline” value before treatment. However, a skilled person will appreciate that the sample may also be obtained after exposure to a BCID or TCID agent, in particular, if the exposure was a low amount and/or occurred several days, weeks, months, or years prior to the determination of the IFN response.

Preferably, the method further comprises comparing the determined level of an IFN response in the sample to a reference. The reference is preferably selected from:
a) a reference value, such as a value obtained from a population, wherein the reference value is obtained from one or more individuals not afflicted with a disease, wherein IFN contributes to the disease, disease activity and/or severity,
b) the level of an IFN response in a second sample from the patient that has been exposed to a BCID or TCID, or
c) the level of an IFN response in a second sample from the patient that has been exposed to an IFN or IFN-inducing agent.

As it will be clear to a person skilled in the art, a second sample may be obtained independently from a first sample or it may be obtained at the same time as the first sample, e.g., where the first sample is split into at least two parts.

The second sample may be exposed to a BCID or TCID or an IFN or IFN-inducing agent either in vitro or in vivo. For example, the second sample may be obtained from the patient after the start of treatment. Preferably, the second sample is exposed in vitro.

The disclosure further provides a method for prognosticating the clinical response to an IFN or IFN-inducing agent in a patient afflicted with a disease, wherein IFN contributes to the disease, disease activity and/or severity and, the method comprising:
providing a sample from the patient, preferably, wherein the patient has not previously been exposed to a B-lymphocyte inhibiting or depleting agent (BCID) or a T-cell inhibiting or depleting agent (TCID),
determining the level of an IFN response in the sample, and
prognosticating the clinical response from the IFN response,

wherein a low level of IFN response indicates the likelihood of a poor clinical response to the IFN or IFN-inducing agent, and

wherein the patient is preferably a candidate for treatment with BCID or TCID.

Preferably, the method further comprises comparing the determined level of an IFN response in the sample to a reference. Preferably, the reference is selected from:
a) a reference value or, preferably, wherein the reference value is obtained from one or more individuals not afflicted with a disease, wherein IFN contributes to the disease, disease activity and/or severity,
b) the level of an IFN response in a second sample from the patient that has been exposed to a BCID or TCID.

Preferably, the IFN response is determined by determining the level of an expression product of a gene selected from the group consisting of genes listed in Tables 1A, 1B, 1C, 1D, and 2, BAFF and DARC. Preferably, the IFN response level is determined by determining the expression level of BAFF and DARC genes supplemented with at least one gene selected from the group consisting of genes from Tables 1A, 1B, 1C, 1D, and 2. Preferably, the IFN response level is determined by determining the level of an expression product of at least one gene selected from the group consisting of Mx1 (MxA), ISG15, OAS1, LGALS3BP, RSAD2, IFI44L, IFI44, Mx2 (MxB), OAS2, DARC, BAFF, HERC5, Ly6E, IFI27, RAP1GAP, EPSTI1 and/or SERPING1. Preferably, the IFN response level is determined by determining the level of an expression product of at least one gene selected from the group consisting of OAS 1 and Mx2. Preferably, the IFN response level is determined by determining the level of an expression product of at least one gene selected from the group consisting of RSAD2 and IFI44L. Preferably, the IFN response level is determined by determining the level of an expression product of at least one gene selected from the group consisting of Mx1, ISG15, OAS2 and SERPING1.

The disclosure further provides a method of treating an individual suffering from or at risk of suffering from a B- and/or T-cell-related disease or from a with a disease, wherein IFN contributes to the disease, disease activity and/or severity with a soluble BCID or TCID agent, comprising the steps of determining a prognosis for a clinical response to a treatment with the soluble BCID or TCID agent as described herein and treating the individual with the soluble BCID or TCID agent, if the individual has been prognosticated as a good responder.

BCIDT represent an important advancement in therapy for chronic inflammatory disease. BCIDT have been implied for B- and T-cell, and auto-antibody-associated autoimmune diseases (AAID) such as multiple sclerosis (S. L. Hauser et al., B-cell depletion with rituximab in relapsing-remitting multiple sclerosis, N Engl. J. Med. 2008, 358; 676-688), Grave's disease (L. Fassi et al., Treatment of Grave's disease with rituximab specifically reduces the production of thyroid stimulating antibodies, Clin. Immunol. 2009, 130:352-358), Wegener's disease, Pemphigus Vulgaris (A. R. Ahmed et al., Treatment of pemphigus vulgaris with rituximab and intravenoud immunoglobulin, N Engl. J. Med. 2006, 54:2970-2982; Mouquet et al., B-cell depletion immunotherapy in pemphigus: effects on cellular and humoral immune responses, J. Invest. Derm. 2008, 128:2859-2869), systemic lupus erythematosus (I. Gunnarsson et al., Histopathologic and clinical outcome of rituximab treatment in patients with cyclophosphamide-resistant proliferative lupus nephritis, Arth. Rheum. 2007, 56:1263-1272; R. A. Guzman et al., Rituximab in refractory systemic lupus erythemathosus, Lupus 2005, 14:221 (OP18)), Sjögren's syndrome (R. A. Guzman et al., Rituximab in primary Sjögren's syndrome, J. Clin. Rheumatol. 2006, 12:164 (s52)), some forms of vasculitis, some types of inflammatory muscle disease (R. A. Guzman et al., B-cell depletion in poly-dermatomyositis, 6^{th} International Congress on Autoimmunity, Porto Portugal 2008 (www.kenes.com/autoimmunity)), systemic sclerosis, type I diabetes and immune and thrombotic thrombocytopenic purpura (R. Stasi et al., Rituximab chimeric anti-CD20 monoclonal antibody treatment for adults with chronic idiopathic thrombocytopaenic purpura, Blood 2001, 98:952-957). However, clinical experience showed that BCIDT is not effective for all but a subset of the patients treated (P. Emery, R. Fleischmann, A. Filipowicz-Sosnowska, J. Schechtman, L. Szczepanski, A. Kavanaugh, et al., the efficacy and safety of rituximab in patients with active rheumatoid arthritis despite methotrexate treatment: results of a phase JIB randomized, double-blind, placebo-controlled, dose-ranging trial, Arthritis Rheum. 2006, 54(5):1390-1400; S. B. Cohen, P. Emery, M. W. Greenwald, M. Dougados, R. A. Furie, M. C. Genovese, et al., Rituximab for rheumatoid arthritis refractory to anti-tumor necrosis factor therapy: Results of a multicenter, randomized, double-blind, placebo-controlled, phase III trial evaluating primary efficacy and safety at twenty-four weeks, Arthritis Rheum. 2006, 54(9):2793-2806; K. Hawker, P. O'Connor, M. S. Freedman, P. A. Calabresi, J. Antel, J. Simon, S. Hauser, E. Waubant, T. Vollmer, H. Panitch, J. Zhang, P. Chin, C. H. Smith, OLYMPUS trial group, Rituximab in patients with primary progressive multiple sclerosis: results of a randomized double-blind placebo-controlled multicenter trial, Ann. Neurol. 2009 October, 66(4):460-471; J. T. Merrill, C. M. Neuwelt, D. J. Wallace, J. C. Shanahan, K. M. Latinis, J. C. Oates, T. O. Utset, C. Gordon, D. A. Isenberg, H. J. Hsieh, D. Zhang, and P. G. Brunetta, Efficacy and safety of rituximab in moderately to severely active systemic lupus erythematosus: the randomized, double-blind, phase II/III systemic lupus erythematosus evaluation of rituximab trial, Arthritis Rheum. 2010 Jan, 62(1):222-233).

The disclosure relates to our finding that demonstrates that BCIDT, more specifically rituximab, induces an increase of IFN bioactivity (preferably, type I), IFN-like bioactivity (preferably, type I) and/or IFN response activity (preferably, type I) in a subset of patients who are treated. IFN was first described by Alick Isaacs and Jean Lindenmann in 1957 (Q. A. Isaacs, J. Lindenmann, Virus interference I, The interferon, Proc. R. Soc. Lond. B. Biol. Sci. 1957, 147:258-267).

Several IFNs have now been identified, which are classified into three families, on the basis of gene sequence, chromosomal location, and receptor specificity (S. Pestka, C. D. Krause, M. R. Walter, Interferons, interferon-like cytokines, and their receptors, Immunol. Rev. 2004, 202:8-32). IFNs are considered to play a crucial role in the antiviral response and both innate immunity and adaptive immunity. Thereto, the IFNs induce the expression of hundreds of genes involved in many biological functions (S. D. Der, A. Zhou, B. R. Williams, et al., Identification of genes differentially regulated by interferon alpha, beta and gamma using ologinucleotide arryas. Proc. Natl. Acad. Sci. USA 1998; 95:15623-15628). Some of these responsive genes are shared between the IFN families, whereas others are specific for a single IFN type or IFN family. Human interferons include IFNA1, IFNA2, IFNA4, IFNA5, IFNA6, IFNA7, IFNA8, IFNA10, IFNA13, IFNA14, IFNA16, IFNA17, IFNA21, IFNB1, IFNW, IFNE1, and IFNK. Preferably, the methods described herein relate to the type I IFN response. Preferably, the methods described herein relate to type I IFNs.

The signature of relevance in the prediction of the response to BCIDT, in particular rituximab, and the pharmacological effects associated with the clinical outcome are primarily of the IFN type I type, i.e., induced by type I IFN-like cytokines such as IFNalpha and IFNbeta. The IFNs (preferably type I) and their specific response programs might exert both immune stimulatory and immune suppressive effects. For example, administration of IFN type I in patients with multiple sclerosis (MS), a chronic inflammatory brain disease, is successfully used to prevent further disease progression. IFNbeta decreases clinical relapses, reduces brain disease activity, and possibly slows down progression of disability (The IFNB Multiple Sclerosis Study Group, Interferon beta-1b is effective in relapsing-remitting multiple sclerosis. I. Clinical results of a multicenter, randomized, double-blind, placebo-controlled trial, Neurology 1993 April, 43(4):655-661; PRISMS (Prevention of Relapses and Disability by Interferon beta-1a Subcutaneously in Multiple Sclerosis) Study Group, randomized double-blind placebo-controlled study of interferon beta-1a in relapsing/remitting multiple sclerosis, Lancet 1998 Nov. 7, 352(9139):1498-504; L. D. Jacobs, D. L. Cookfair, R. A. Rudick, R. M. Herndon, J. R. Richert, A. M. Salazar, et al., Intramuscular interferon beta-1a for disease progression in relapsing multiple sclerosis, The Multiple Sclerosis Collaborative Research Group (MSCRG), Ann. Neurol. 1996 March, 39(3):285-94.).

Conversely, type I IFN and type I IFN-induced genes were found to play a role in the pathogenesis of connective tissue diseases such as systemic lupus erythematosus (SLE), Sjögren's disease (SS), polymyositis and systemic sclerosis (SSc). Moreover, patients treated with IFNalpha or IFNbeta often produce antinuclear auto-antibodies, which may lead to development of lupus in approximately 1% of the patients treated, which observation confirms the propensity of IFNalpha in inducing SLE. Thus, whereas IFNs (preferably type I) are known to have beneficial effects in one class of chronic inflammatory diseases such as MS, type I IFNs have a detrimental and disease-inducing effect in another class of disease such as SLE. SLE is a chronic inflammatory autoimmune disease characterized by the production of auto-antibodies with specificity to a series of nuclear antigens (D. J. Wallace, Dubois' Lupus Erythematosus (Williams & Wilkins, Philadelphia) (2002)). Serum levels of IFNalpha in patients with SLE were reported to be increased and to correlate with disease activity parameters such as SLEDAI, number of organs involved, titer of anti-dsDNA antibodies, and degree of hypocomplementemia (A. A. Bengtsson, G. Sturfelt, L. Truedsson et al., Activation of the type I interferon system in systemic lupus eruthematosus correlates with disease activity but not with antiretroviral antibodies, Lupus 2000; 9:664-671).

Several diseases are currently known to benefit from BCIDT, such as rituximab. The term “patient” refers to any subject (preferably human) afflicted with a disease likely to benefit from BCIDT, in particular, a B-cell-related disease. B-cells are the precursors of antibody-producing cells (plasma cells). In the process of undergoing activation and maturation into memory B-cells and plasma cells, they are very efficient antigen-presenting cells (APCs) to T-cells of soluble antigens that are bound specifically by the B-cell antigen receptor (surface immunolglobuline). B-cell ontogeny is characterized by a series of changing surface phenotypes. One of these is the CD20 surface marker (a 33-37 kDa membrane-associated phosphoprotein) expressed during intermediate stages of development, which is lost during terminal differentiation to the immunoglobulin-producing plasma cell. The exclusivity and high specificity of B-cell molecules like CD20 make these types of proteins attractive pharmaceutical targets.

A specific beneficial feature for CD20 is that free CD20 is not present in the circulation, CD20 does not modulate its own expression, and is not shed or internalized after antibody binding. Moreover, no endogeneous CD20-like molecules are known that interfere with its function (Press et al., Monoclonal antibody 1F5 (anti-CD20) serotherapy of B-cell lymphomas, Blood 1987, 69:584-591). Diseases wherein B-cells directly contribute to pathogenesis and/or indirectly influence disease via changes in T-cell function can be efficiently treated with BCIDT. B-cell targeting via anti-CD20, e.g., rituximab (an anti-CD20 antibody), rapidly depletes peripheral blood CD20-positive B-cells via complement-mediated and antibody-dependent cell-mediated cytotoxicity (ADCC), induction of apoptosis and inhibition of cell growth (D. G. Maloney et al., Rituximab: Mechanism of action and resistance, Semin. Oncol. 2002, 29:2-9). B-cell levels usually reach a minimum by one month and repopulation generally starts by six months. Rituximab also down-regulates CD40 ligand, CD40 and CD80, resulting in changes to T-cell function (M. Tokunaga et al., Down-regulation of CD40 and CD80 on B-cells in patients with life-threatening systemic lupus erythematosus after successful treatment with rituximab, Rheumatology 2005, 44:176-182).

Thus, despite the overall decrease in the expression of B-cell markers in patients treated with rituximab, marked variability between individual clinical responses have been observed between patients, with a portion of patients failing to achieve a favorable clinical response and others who reach a clinical benefit or remission. The clear contrasting results concerning the efficacy of rituximab between patients leaves open the question what the mechanism of action is for rituximab and what discerns responders from non-responders. Given the destructive nature of chronic inflammatory diseases, risk of adverse effects and considerable costs for therapy and alternative treatment options that have become available, there is a strong need to make predictions on clinical success before start of therapy. Thus, it is highly desirable to predict whether a patient will respond to BCIDT and to find markers to be able to follow the clinical efficacy during therapy. To accomplish the goal of biomarker-driven prediction and monitoring of clinical response, consideration of the molecular and immunological variables that might influence this therapy and help inform clinical practice and future studies is important.

Described herein is a modulating effect of rituximab on IFN type I activity that may have concomitant beneficial or detrimental effects, depending on the disease. For diseases like MS and RA, the pharmacological induction of type I IFN-activity could be an important factor in the ameliorative effect of B-cell depletion therapy. It is now well established that IFNbeta products show clinical efficacy in relapsing-remitting MS. For RA, such a role of type I IFN activity is highlighted by Treschow et al. (A. P. Treschow, I. Teige, K. S, Nandakumar, et al., Stromal cells and osteoclasts are responsible for exacerbated collagen-induced arthritis in interferon-beta-deficient mice, Arthritis. Rheum. 2005, 52:3739-3748) who showed that IFNβ deficiency prolonged experimental arthritis. Additional evidence for a beneficial effect of type I IFNs in RA has been provided by de Hooge et al. (A. S. de Hooge, F. A. van de Loo, M. I. Koenders et al., Local activation of STAT-1 and STAT-3 in the inflamed synovium during zymosan-induced arthritis: exacerbation of joint inflammation in STAT-1 gene-knockout mice, Arthritis Rheum. 2004, 50:2014-2023) who demonstrated that STAT-1 deficiency resulted in exacerbation of experimental arthritis. Moreover, transfer of IFN-competent FLS was able to ameliorate arthritis in IFNβ-deficient recipients (A. P. Treschow, I. Teige, K. S, Nandakumar et al., Stromal cells and osteoclasts are responsible for exacerbated collagen-induced arthritis in interferon-beta-deficient mice, Arthritis Rheum. 2005, 52:3739-3748). However, although treatment with recombinant-IFNβ revealed promising results in experimental arthritis, treatment of RA patients with IFNβ has been unsuccessful so far, which may be due to issues with dosing and pharmacokinetics (H. J. van, C. Plater-Zyberk, P. P. Tak, Interferon-beta for treatment of rheumatoid arthritis? Arthritis Res. 2002, 4:346-352).

However, in diseases such as SLE, where type I IFN is known to induce and increase disease progression and severity, an increase in IFN (preferably type I) activity during treatment with rituximab will not contribute to the ameliorative effects of B-cell depletion and/or inhibition. Hence, patients who develop an increase in the IFN (preferably type I) activity during rituximab therapy with concomitant disease-promoting effects, may experience a neutralization of the ameliorative effects of BCIDT and even aggravation of disease severity and activity.

For patients where IFN (preferably type I) activity during therapy remains relatively stable, the neutralizing and/or aggravating effect due to an increase in IFN (preferably type I) activity is absent or may be minimal, resulting in an ameliorative effect of BCIDT. The increase in IFN response activity may be related to a relatively low or absent IFN (preferably type I) activity at baseline. A high level of IFN (preferably type I) activity at baseline may result in an absent or relatively low increase of IFN (preferably type I) activity due to the fact that the response is already (almost) saturated. The saturation and/or desensitization of individuals with a high IFN (preferably type I) response activity is based on findings with patients with RRMS who were treated with IFNbeta (L. G. van Baarsen, S. Vosslamber, M. Tijssen, J. M. C. Baggen, L. F. van der Voort, J. Killestein, T. C. van der Pouw Kraan, C. H. Polman, and C. L. Verweij, Pharmacogenomics of Interferon-B therapy in multiple sclerosis: Baseline IFN signature as a biomarker for pharmacological differences between patients, PLoS ONE e1927 (2008)). These findings revealed that a high IFN response activity at baseline may result in an absence or relatively low increase in IFN activity. Thus, a high baseline level in SLE may result in an absent or relative low increase in IFN response activity, thereby eliminating or minimizing the unfavorable IFN-inducing (preferably type I) effects of rituximab during therapy.

Alternatively, a low level of IFN (preferably type I) activity at baseline may result in a relatively large increase in IFN (preferably type I) response activity, which contributes to disease severity and activity resulting in disease aggravation. These findings allow prediction and monitoring the clinical response for BCIDT, such as rituximab, making use of measuring features associated with the IFN (preferably type I) bioactivity, (preferably type I) IFN-like activity and/or IFN (preferably type I) response activity. Therefore, SLE patients are likely to fully benefit from the B-cell depleting or inhibiting effect of BCIDT if they could be stratified in responders and non-responders based on the relative increase in their IFN (preferably type I) bioactivity, IFN-like bioactivity and/or IFN response activity prior to the start of therapy or during therapy.

In a preferred embodiment, the patient suffers from a disease selected from the group consisting of a B- or T-cell-related disease, and an auto-antibody-associated autoimmune disease (AAID), wherein IFN (preferably type I) is a driving factor in the disease susceptibility, disease severity and disease progression. These diseases are likely to benefit from BCIDT.

Preferred diseases are selected from the group consisting of multiple sclerosis, systemic lupus erythematosus, Sjögren's syndrome, some forms of vasculitis, some types of inflammatory muscle disease, systemic sclerosis, type I diabetes and immune and thrombotic thrombocytopenic purpura, and transplant rejection or graft-versus-host disease, malignancy, a pulmonary disorder, an intestinal disorder, a cardiac disorder, a spondyloarthropathy, a metabolic disorder, anemia, pain, a hepatic disorder, and a skin disorder. In one embodiment, the autoimmune disorder is selected from the group consisting of rheumatoid arthritis, rheumatoid spondylitis, osteoarthritis, gouty arthritis, allergy, multiple sclerosis, autoimmune diabetes, autoimmune uveitis, and nephrotic syndrome. In another embodiment, the B- and T-cell, and auto-antibody-associated autoimmune diseases are selected from the group consisting of inflammatory bone disorders, bone resorption disease, periodontal disease. In still another embodiment, the B- and T-cell, and auto-antibody-associated autoimmune diseases are selected from the group consisting of Behcet's disease, ankylosing spondylitis, asthma, chronic obstructive pulmonary disorder (COPD), idiopathic pulmonary fibrosis (IPF), restenosis, diabetes, anemia, pain, a Crohn's disease-related disorder, juvenile rheumatoid arthritis (JRA), psoriatic arthritis, and chronic plaque psoriasis.

In one embodiment of the invention, the B- and T-cell and auto-antibody-associated autoimmune disease is Crohn's disease. In another embodiment, the disease is ulcerative colitis. In still another embodiment, the disease is psoriasis. In still another embodiment, the disease is psoriasis in combination with psoriatic arthritis (PsA).

In another preferred embodiment, the B- and T-cell, and auto-antibody-associated autoimmune disease comprises a disease that is likely to benefit from BCIDT. Preferably, the disease comprises type I diabetes.

The methods described herein are especially useful for predicting responses to treatments in patients afflicted with a disease, wherein IFN contributes to the disease activity and/or severity. Preferably, the disease is selected from systemic lupus erythematosus, Sjögren's disease, myositis, dermatomyositis, polymyositis and systemic sclerosis. More preferably, the disease is selected from systemic lupus erythematosus, Sjögren's disease, polymyositis and systemic sclerosis.

In a preferred embodiment, the patient is an individual suffering from or at risk of suffering from “systemic lupus erythematosus (SLE).” With the term “an individual suffering from or at risk of suffering from SLE” is meant an individual who is diagnosed with SLE or is suspected by a doctor of suffering from SLE or of developing the symptoms of SLE within 10 years. SLE is a systemic inflammatory disease that is characterized by a relapsing and remitting course with flares of high morbidity. The disease predominates in women and may present with severe acute illness characterized by seizures, psychosis, profound anemia, renal failure, pulmonary hemorrhage or sepsis. To fulfill the diagnosis, patients have to fulfill 4 out of 11 criteria (E. M. Tan, A. S. Cohen, J. F. Fries, A. T. Masi, D. J. McShane, N. F. Rothfield, et al., The 1982 revised criteria for the classification of systemic lupus eruthematosus, Arthritis Rheumatism 1988, 31:315-324). Presence of anti-nuclear auto-antibodies (against, e.g., ds DNA, histones, nucleosomes, RNP and Sm), which is observed in approximately 95% of the patients, constitute one of the criteria. The presence of auto-antibodies to cell surface molecules are also often observed and associated with development of thromboembolic complications, hemolytic anemia, neutropenia, thrombocytopenia and severe kidney disease (D. J. Wallace (2002), Dubois' Lupus Erythematosus (Williams & Wilkins, Philadelphia). The disease severity, broad range of clinical involvement and response to treatment is highly variable between patients and poses considerable challenges in the management of lupus. Most of the patients with SLE display an elevated serum level of type I IFNS (IFNalpha and/or beta).

The effects of the type I IFNs may explain many disease features observed in SLE. The increased level of type I IFN response gene activity with concomitant increased expression of neutrophil-related genes correlates with disease activity. Treatment with high dose steroids abrogates the type I IFN signature and induces a good clinical response or remission (L. Bennet, A. K. Palucka, E. Arce, V. Cantrell, J. Borvak, J. Banchereau, V. Pascual, Interferon and granulopoiesis signatures in systemic lupus erythematosus, J. Exp. Med. 2003, 197:711-723).

There is no permanent cure for SLE. Treatment is aimed to relieve symptoms and protect organs by decreasing inflammation and/or the level of autoimmunity. Many patients with mild symptoms may need no treatment or only intermittent courses of anti-inflammatory medications. Those with more serious illness involving damage to internal organ(s) may require high doses of corticosteroids in combination with other medications that have immune-suppressive agents. Non-steroidal anti-inflammatory drugs (NSAIDs) are helpful in reducing inflammation and pain in muscles, joints, and other tissues, such as, aspirin, ibuprofen, naproxen, and sulindac, but responses to NSAIDs vary. Corticosteroids are more potent than NSAIDs in immune suppression, especially when the disease is active. Corticosteroids are particularly helpful when internal organs are affected. Hydroxychloroquine (Plaquenil) is an antimalarial medication found to be particularly effective for SLE people with fatigue, skin involvement, and joint disease. It prevents flare-ups of lupus and significantly decreases the frequency of abnormal blood clots. Plaquenil is commonly used in combination with other treatments for lupus. For resistant skin disease, other antimalarial drugs, such as chloroquine (Aralen) or quinacrine, are considered and can be used in combination with hydroxychloroquine. Alternative medications for skin disease include dapsone and retinoic acid (Retin-A). Retin-A is often effective for an uncommon wart-like form of lupus skin disease.

For more severe skin disease, immunosuppressive medications are considered as described below. Cytotoxic immunosuppressive medications (methotrexate, azathioprine, cyclophosphamide, chlorambucil and cyclosporine) are used for treating people with more severe manifestations of SLE, such as damage to internal organ(s). Most therapies have side effects that are specific for each drug. In recent years, mycophenolate mofetil has been used as an effective medication for lupus, particularly when it is associated with kidney disease. In SLE patients with serious brain or kidney disease, plasmapheresis (a process of removing and treating the blood before it is returned to the body) is sometimes used to remove antibodies and other immune substances from the blood. Most recent research is indicating benefits of rituximab (Rituxan) in treating lupus. Rituximab is an intravenously infused antibody that suppresses a particular white blood cell, the B-cell, by decreasing their number in the circulation.

With the term “clinical response” is meant the clinical result of BCIDT. The clinical response can be a positive or a negative clinical response. With a “positive clinical response” is meant that the severity of symptoms or the number of symptoms is reduced as a result of BCIDT or TCIDT. There are several lupus activity and outcome measurements (M. Petri, Disease activity assessment in SLE: do we have the right instruments? Ann. Rheum. Dis. 2007, 66:61-64). Each has strengths and weaknesses; none is perfect. Most are global disease activity indices, giving a summary score of the entirety of SLE activity. Two of the indices, the Systemic Lupus Erythematosus Disease Activity Index (SLEDAI) and the British Isles Lupus Activity Group (BILAG) index, have been the predominant ones used in randomized clinical trials. The BILAG is organ-specific, but can also be converted into a global score. It requires the physician to score organ manifestations as improved (1), same (2), worse (3) or new (4) since the last month. Multiple organ manifestations and/or laboratory tests are combined into a single score for that organ, which range from A (“active”), B (“beware”), and C (“contentment”) when there is activity, and D (“resolved activity”) till E (“never involved”) when there is not. The SLEDAI consists of a list of defined organ manifestations, which are “present” or “absent” during the last 10 days. It is a weighted instrument in which descriptors are multiplied by that organ's “weight.” These weighted organ manifestations are added-up into the final score. To be useful in clinical trials, they must also be able to demonstrate change over time. Some activity indices include patient-derived assessments, including BILAG and the Systemic Lupus Activity Measure (SLAM). Also, the concept that SLE has “flared”: an increase in activity over a defined amount of time is used to measure outcome. This concept of flare has been defined using the existing disease activity indices. Using as a gold standard a 1.0 increase on a 0 to 3 visual analogue scale, “flare” corresponded to a change in 3 points or more on SLAM, 3 points or more on the SLEDAI, or 4 points or more on a global BILAG.

Preferably, the clinical outcome is the result of a treatment with a soluble BCIDT. When the disease is SLE, it is preferred that a positive clinical response comprises at least reduction of swelling of joints (SLEDAI, BILAG or SLAM). Preferably, an assessment of a clinical response is based on standardized and preferably validated clinical response criteria such as provided by the guidelines of organizations such as the National Institute for Health and Clinical Excellence (NICE), EULAR and/or ACR. Even more preferably, clinical response criteria are combined with demographic data, other clinical information or information about relevant habits. Demographic data comprise gender and/or age. Clinical information may comprise any relevant clinical observation or data. Preferred clinical information comprises anti/DNA, complement, anti nuclear antigen antibodies (ANA), CRP, ESR, disease duration and medication. Information about relevant habits may be any relevant information.

B-lymphocyte dysregulation with the production of autoantibodies, formation of immune complexes and release of destructive mediators are known to contribute to SLE pathogenesis (M. Mannik and F. A. Nardella, IgG rheumatoid factors and self-association of these antibodies, Clin. Rheum. Dis. 1985, 11:551-572). Approximately 90% of the SLE patients develop autoantibodies produced by B-cells. B-cells carry out central roles in the pathogenesis of SLE through a combination of antibody-mediated and antibody-independent actions. These actions include the presentation of autoantigens, induction of CD4+ helper T-cells and CD8+ effector T-cells, maintenance of T-cell memory, inhibition of regulatory T-cells (TREG), secretion of pro-inflammatory cytokines and chemokines, and organization of tertiary lymphoid tissue, all of which might promote the generation and/or amplification of autoimmune responses in target organs (N. Manjarrez-Orduno, T. D. Quach, and I. Sanz, B-cells and immunological tolerance, J. Invest. Dermatol. 129:278-288 (2009); K. Yanaba, et al., B-lymphocyte contributions to human autoimmune disease, Immunol. Rev. 223:284-299 (2008); and F. E. Lund, Cytokine-producing B lymphocytes—key regulators of immunity, Curr. Opin. Immunol. 20:332-338 (2008)).

This insight, together with the success of B-cell depletion for the treatment of rheumatoid arthritis (RA), started investigations of B-cell depletion in SLE almost 10 years ago. Indeed, B-cell-depleting therapy with the monoclonal antibody rituximab, directed against the B-cell-specific antigen CD20, has in recent years shown encouraging results in patients with SLE (J. H. Anolik, J. Barnard, A. Cappione, A. E. Pugh-Bernard, R. E. Felgar, R. J. Looney, et al., Rituximab improves peripheral B-cell abnormalities in human systemic lupus erythematosus, Arthritis Rheum. 2004, 50:3580-3590; M. J. Leandro, G. Cambridge, J. C. Edwards, M. R. Ehrenstein, D. A. Isenberg, B-cell depletion in the treatment of patients with systemic lupus erythematosus: a longitudinal analysis of 24 patients, Rheumatology (Oxford) 2005, 44:1542-1545; R. J. Looney, J. H. Anolik, D. Campbell, R. E. Felgar, F. Young, L. J. Arend, et al., B-cell depletion as a novel treatment for systemic lupus erythematosus: a phase I/II dose-escalation trial of rituximab, Arthritis Rheum. 2004, 50:2580-2589; R. F. van Vollenhoven, I. Gunnarsson, E. Welin-Henriksson, B. Sundelin, A. Osterborg, S. H. Jacobson, et al., Biopsy-verified response of severe lupus nephritis to treatment with rituximab (anti-CD20 monoclonal antibody) plus cyclophosphamide after biopsy-documented failure to respond to cyclophosphamide alone, Scand. J. Rheumatol. 2004, 33:423-427). However, randomized, placebo-controlled trials of rituximab failed to meet their primary or secondary clinical endpoints for renal and nonrenal SLE (J. T. Merrill et al., Efficacy and safety of rituximabin moderately to severely active systemic lupus erythematosus: The randomized, double-blind, phase ii/iii systemic lupus erythematosus evaluation of rituximab trial, Arthritis Rheum. 62:222-233 (2010)). In these studies, the investigators were unable to show that rituximab was clinically superior to placebo when added to standard care in contrast to the existence of numerous observational studies that showed efficacy. These unexpected findings have caused confusion and resulted in a need to understand the pharmacological effects and approaches to explore and use biomarkers to discriminate between responders and non/responders for rituximab. Therefore, strategies should be installed to select those patients who will respond to therapy and monitor the therapy response.

In order to do so, we performed pharmacogenomic analyses in patients with RA who were treated with rituximab. These studies demonstrated that despite the overall decrease in the expression of B-cell markers, RA patients exhibited interindividual differences in their pharmacological responses upon rituximab therapy. Among these, we observed a clear difference in the kinetics of only one gene signature during rituximab treatment between responders and non-responders. This signature represents type I IFN-response genes. With regard to pharmacodynamics of rituximab in relation to the type I IFN activity, two interesting observations were made. First, non-responder RA patients already displayed an activated type I IFN system before the start of treatment, which remains active during treatment. Second, good responder RA patients have low or absent IFN response activity at baseline and develop IFN response activity during three months of therapy that is comparable to that of the non-responders. The differential response correlated with baseline levels of IFN response genes, which were significantly lower in responders compared to non-responders. These findings lead us to conclude that an increase in IFN response activity during rituximab treatment is associated with the biological mechanism underlying the therapeutic response in RA.

Factors known to induce IFNs (preferably type I) and the consecutive induction of IFN response activity consist of exogenous (infectious) agents and endogenous agents, such as nucleic acids and apoptotic/necrotic material. Hence, subsequent release from apoptotic/necrotic material from depleted CD20+ B-cells may promote IFN production and release, which might selectively take place in the IFN^{low} patients. Thus, depletion of any cell-subset by antibodies such as rituximab (anti-CD20), which leads to complement-mediated lysis, antibody-dependent cell-mediated cytotoxicity (ADCC), and/or apoptosis, leads to release of cellular/nuclear substances in the circulation. Subsequently, these agents trigger Toll-Like Receptors (TLR) or cytosolic DNA and/or RNA sensors on and/or in immune cells, which results in the release of bioactive IFN type I or IFN type I-like agents that are responsible for the induction of a type I IFN response activity in the blood.

Thus, the release of such endogenous TLR ligands (e.g., RNA, DNA, HMGB1, HSPs, Fatty acids, Hyaluronan fragments, Fibronectin fragments, Immune complexes/dsDNA, Immune complexes/RNA, MRp 8 and 14 (Stefan K. Drexler, Brian M. Foxwell, The role of Toll-like receptors in chronic inflammation, The International Journal of Biochemistry & Cell Biology (2010) 42:506-518)) is a likely cause of the induction of IFN type I response activity, which could be beneficial in diseases such as MS and RA and detrimental in, e.g., SLE. Moreover, the relative extent of the increase in IFN (preferably type I) response activity is determined by the IFN (preferably type I) response activity at baseline. In this scenario, every cell-depleting agent is able to induce an IFN (preferably type I) response activity.

The extent and induction of the response may depend on genetic factors in TLR/IFN regulatory genes such as IRF5 (R. R. Graham, S. V. Kozyrev, E. C. Baechler, M. V. Reddy, R. M. Plenge, J. W. Bauer, et al., A common haplotype of interferon regulatory factor 5 (IRF5) regulates splicing and expression and is associated with increased risk of systemic lupus erythematosus, Nat. Genet. 2006 May, 38(5):550-555; S. Sigurdsson, G. Nordmark, H. H. Goring, K. Lindroos, A. C. Wiman, G. Sturfelt, et al., Polymorphisms in the tyrosine kinase 2 and interferon regulatory factor 5 genes are associated with systemic lupus erythematosus, Am. J. Hum. Genet. 2005 March, 76(3):528-537) and/or the sensitization/desensitization of the cellular system, as we observed in multiple sclerosis (L. G. van Baarsen, S. Vosslamber, M. Tijssen, J. M. C. Baggen, L. F. van der Voort, J. Killestein, T. C. van der Pouw Kraan, C. H. Polman, C. L. Verweij, Pharmacogenomics of Interferon-β therapy in multiple sclerosis: Baseline IFN signature as a biomarker for pharmacological differences between patients, PLoS ONE e1927 (2008)). The fact that the IFN response activity doesn't increase in the IFN^{high} patients might be explained by a saturated and desensitized IFN system as was previously observed in a subset of patients with multiple sclerosis who are insensitive to the pharmacological and clinical effects of IFNβ treatment.

The term “BCIDT” refers to molecules, such as proteins or small molecules, that can significantly reduce B-cell function and/or number, and/or T-cell function. Preferably, the BCIDT comprise anti-B-cell antibodies, e.g., rituximab (Chimeric IgG1 Genentech/Biogen Approved 1997), Y⁹⁰-Ibritumomab tiuxetan (Murine (⁹⁰Y) NHL Biogen/IDEC Low ADCC Approved 2002), I¹³¹tositumomab (Murine (131I) NHL GSK Low CDC Approved 2003), Ofatumumab (Human IgG1 NHL/RA Genmab AC/GSK High CDC and ADCC Phase III trials), Ocrelizumab (Humanized IgG1 NHL/RA Genentech/Roche/Biogen Phase III trials), TRU-015 (SMIP# RA Trubion Pharma/Wyeth High ADCC Phase I/II Low CDC), Veltuzumab (Humanized NHL and ITP Immunomedics Phase I/II IgG1), AME-133v (Humanized IgG1 Relapsed NHL Applied Molecular High ADCC Phase I/II Evolution/Eli Lilly), PRO131921 (Humanized IgG1 CLL and NHL Genentech High CDC and ADCC Phase I/II (Version 114)), GA10168 (Humanized CLL and NHL Glycart/Roche High PCD and ADCC Phase I/II), and anti-T-cell antibodies, e.g., Abatacept (recombinant fusion protein that selectively modulates CD80 and CD86-CD28 costimulatory signal required for full T-cell activation), and alefacept (bivalent recombinant fusion protein consisting of a LFA-3 portion that binds CD2 receptors on T-cells, IgG1 portion of alefacept binds to Fc-R receptor on natural killer cells to induce T-cell apoptosis). In fact, all therapies that target B-cell (e.g., CD19, BAFF receptor) and T-cell surface markers fall in this category. Most preferably, the BCID is rituximab.

Preferred therapies with soluble B- and T-cell inhibitory or depleting molecules of the invention include, for example, rituximab, Y⁹⁰-Ibritumomab tiuxetan, I¹³¹tositumomab, Ofatumumab, Ocrelizumab, and anti-T cell antibodies, e.g., abatacept, and alefacept. More preferably, the soluble B- and T-cell inhibitory or depleting molecule comprises rituximab.

With the term “sample” is meant any suitable sample comprising proteins or nucleotides. Preferred suitable samples include whole blood, saliva, fecal material, buccal smears, skin, and biopsies of specific organ tissues, such as muscle or nerve tissue and hair follicle, because these samples comprise relevant expression products. Preferably, the cell sample is a blood sample because a blood sample is easily obtainable and comprises large amounts of relevant expression products. Preferably, the sample comprises cells and serum/plasma. Preferably, the sample is from a patient who has not received treatment with a soluble BCID or TCID agent. Preferably, a second sample is from an individual between 1 and 8 months after the first exposure of the individual to the soluble BCID or TCID agent. Preferably, a second sample is also taken at baseline, preferably simultaneously with sample one prior to the start of therapy, has been exposed in vitro to the soluble BCID or TCID agent. As it will be clear to a person skilled in the art, a second sample may be obtained independently from a first sample or it may be obtained at the same time as the first sample, e.g., where the first sample is split into at least two parts. Preferably, a second sample is taken at baseline, preferably simultaneously with sample one prior to the start of therapy, and is exposed in vitro to IFN or an IFN-inducing agent such as dsDNA or dsRNA. Preferably, a second sample is provided from a patient that has been exposed to a BCID or TCID agent.

The term “IFN response” as used herein refers to a type I, type II, or type III-like bioactivity and/or the expression product of a gene or series of products of genes that become activated in response to type I, H, or III IFN bioactivity or IFN-like bioactivity or genes involved in the IFN type I, II, or III pathway, respectively. With the “level of an IFN-type response” is meant the amount of expression product of any gene or its product involved in the IFN response pathway. The term “IFN response” also includes the expression of an IFN. The determination of the level of an IFN response, therefore, may also include the determination of the expression of an interferon, for example, in the blood of a patient.

The IFN response pathways overlap, in particular, if there is a significant overlap in the IFN I and IFN III response pathway. In blood cells, the IFN I and IFN III response is nearly identical.

With the term “IFN I-type response” is meant a type I IFN, a type I IFN-like bioactivity and/or the expression product of a gene or series of products of genes that become activated in response to type I IFN bioactivity or type I IFN-like bioactivity or genes involved in the IFN type I pathway. With the “level of an IFN I type response” is meant the amount of expression product of any gene or its product involved in the IFN I response pathway.

As used herein, an IFN-inducing agent is an agent that leads to the significant induction in IFN or induction of an IFN response. IFN-inducing agents are known in the art and include dsDNA, dsRNA, cell debris, cytokines, such as interleukin-1, interleukin-2, interleukin-12, tumor necrosis factor and colony-stimulating factor, autoantibodies, and immune complexes.

An “expression product” of a gene is RNA produced from the genes or a protein produced from the RNA. The levels of the expression products may be determined separately for each different expression product or as a single measurement for more different expression products simultaneously. Preferably, the determination of the level of the expression products is performed for each different expression product separately, resulting in a separate measurement of the level of the expression product for each different expression product. This enables a more accurate comparison of expression levels of expression products with the expression levels of the same expression products in a control.

Determination of the level of the expression products according to methods of the invention may comprise the measurement of the amount of nucleic acids or of proteins. In a preferred embodiment of the invention, determination of the level of the expression products comprises determination of the amount of RNA, preferably mRNA. A level can be the absolute level or a relative level compared to the level of another mRNA. mRNA can be isolated from the samples by methods well known to those skilled in the art as described, e.g., in Ausubel et al. (Current Protocols in Molecular Biology, Vol. 1, pp. 4.1.1-4.2.9 and 4.5.1-4.5.3, John Wiley & Sons, Inc. (1996)). Methods for detecting the amount of mRNA are well known in the art and include, but are not limited to, northern blotting, reverse transcription PCR, real-time quantitative PCR and other hybridization methods. The amount of mRNA is preferably determined by contacting the mRNAs with at least one sequence-specific oligonucleotide that hybridizes to the mRNA.

In a preferred embodiment, the mRNA is determined with two sequence-specific oligonucleotides that hybridize to different sections of the mRNA. The sequence-specific oligonucleotides are preferably of sufficient length to specifically hybridize only to the RNA or to a cDNA prepared from the mRNA. As used herein, the term “oligonucleotide” refers to a single-stranded nucleic acid. Generally, the sequence-specific oligonucleotides will be at least 15 to 20 nucleotides in length, although in some cases, longer probes of at least 20 to 25 nucleotides will be desirable. The sequence-specific oligonucleotides may also comprise non-specific nucleic acids. Such non-specific nucleic acids can be used for structural purposes, for example, as an anchor to immobilize the oligonucleotides.

The sequence-specific oligonucleotide can be labeled with one or more labeling moieties to permit detection of the hybridized probe/target polynucleotide complexes. Labeling moieties can include compositions that can be detected by spectroscopic, biochemical, photochemical, bioelectronic, immunochemical, and electrical optical or chemical means. Examples of labeling moieties include, but are not limited to, radioisotopes, e.g., 32P, 33P, 35S, chemiluminescent compounds, labeled binding proteins, heavy metal atoms, spectroscopic markers such as fluorescent markers and dyes, linked enzymes, mass spectrometry tags, and magnetic labels. Oligonucleotide arrays for mRNA or expression monitoring can be prepared and used according to techniques that are well known to those skilled in the art as described, e.g., in Lockhart et al. (Nature Biotechnology, Vol. 14, pp. 1675-1680 (1996); McGall et al., Proc. Natl. Acad. Sci. USA, Vol. 93, pp. 13555-13460 (1996); and U.S. Pat. No. 6,040,138).

A preferred method for determining the amount of mRNA involves hybridization of labeled mRNA to an ordered array of sequence-specific oligonucleotides. Such a method allows the simultaneous determination of the mRNA amounts. The sequence-specific oligonucleotides utilized in this hybridization method typically are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc.

In one embodiment, determining the level(s) of the expression products is performed by measuring the amount of protein. The term “protein” as used herein may be used synonymously with the term “polypeptide” or may refer to, in addition, a complex of two or more polypeptides that may be linked by bonds other than peptide bonds, for example, such polypeptides making up the protein may be linked by disulfide bonds. The term “protein” may also comprehend a family of polypeptides having identical amino acid sequences but different post-translational modifications, particularly as may be added when such proteins are expressed in eukaryotic hosts. These proteins can be either in their native form or they may be immunologically detectable fragments of the proteins resulting, for example, from proteolytic breakdown. By “immunologically detectable” is meant that the protein fragments contain an epitope that is specifically recognized by, e.g., mass spectrometry or antibody reagents as described below. Protein levels can be determined by methods known to the skilled person comprising, but not limited to: mass spectrometry, Western blotting, immunoassays, protein expression assay, protein microarray, etc. In a preferred embodiment, the level of interferon protein is determined, preferably in a blood sample. Preferably, the interferon is a type I interferon, such as IFNalpha and/or IFNbeta.

One embodiment provides a protein microarray (Templin et al. 2004, Comb. Chem. High Throughput Screen., vol. 7, no. 3, pp. 223-229) for simultaneous binding and quantification of the at least two biomarker proteins according to the invention. The protein microarray consists of molecules (capture agents) bound to a defined spot position on a support material. The array is then exposed to a complex protein sample. Capture agents such as antibodies are able to bind the protein of interest from the biological sample. The binding of the specific analyte proteins to the individual spots can then be monitored by quantifying the signal generated by each spot (MacBeath 2002, Nat. Genet., vol. 32 Suppl. pp. 526-532; Zhu & Snyder 2003, Curr. Opin. Chem. Biol., vol. 7, no. 1, pp. 55-63).

Protein microarrays can be classified into two major categories according to their applications. These are defined as protein expression microarrays and protein function microarrays (Kodadek 2001, Chem. Biol., vol. 8, no. 2, pp. 105-115). Protein expression microarrays mainly serve as an analytic tool, and can be used to detect and quantify proteins, antigen or antibodies in a biological fluid or sample. Protein function microarrays on the other hand can be used to study protein-protein, enzyme-substrate and small molecule-protein interactions (Huang 2003, Front Biosci., vol. 8, p. d559-d576). Protein microarrays also come in many structural forms. These include two-dimensional microarrays constructed on a planar surface, and three-dimensional microarrays that use a flow-through support.

Types of protein microarray set-ups include reverse phase arrays (RPAs) and forward phase arrays (FPAs) (Liotta et al. 2003, Cancer Cell, vol. 3, no. 4, pp. 317-325). In RPAs, a small amount of a tissue or cell sample is immobilized on each array spot, such that an array is composed of different patient samples or cellular lysates. In the RPA format, each array is incubated with one detection protein (e.g., antibody), and a single analyte endpoint is measured and directly compared across multiple samples. In FPAs capture agents, usually an antibody or antigen are immobilized onto the surface and act as a capture molecule. Each spot contains one type of immobilized antibody or capture protein. Each array is incubated with one test sample, and multiple analytes are measured at once.

One of the most common forms of FPAs is an antibody microarray. Antibody microarrays can be produced in two forms, either by a sandwich assay or by direct labeling approach. The sandwich assay approach utilizes two different antibodies that recognize two different epitopes on the target protein. One antibody is immobilized on a solid support and captures its target molecule from the biological sample. Using the appropriate detection system, the labeled second antibody detects the bound targets. The main advantage of the sandwich assay is its high specificity and sensitivity (Templin, Stoll, Bachmann, & Joos 2004, Comb. Chem. High Throughput Screen., vol. 7, no. 3, pp. 223-229). High sensitivity is achieved by a dramatic reduction of background yielding a high signal-to-noise ratio. In addition, only minimal amounts of labeled detection antibodies are applied in contrast to the direct labeling approach where a huge amount of labeled proteins are present in a sample. The sandwich immunoassay format can also be easily amenable to the field of microarray technology, and such immunoassays can be applied to the protein microarray format to quantify proteins in conditioned media and/or patient sera (Huang et al. 2001, Clin. Chem. Lab Med., vol. 39, no. 3, pp. 209-214; Schweitzer et al. 2002, Nat. Biotechnol., vol. 20, no. 4, pp. 359-365).

In the direct-labeling approach, all proteins in a sample are labeled with a fluorophore. Labeled proteins that bind to the protein microarray, such as to an antibody microarray, are then directly detected by fluorescence. An adaptation of the direct-labeling approach is described by Haab and co-workers (Haab, Dunham, & Brown 2001, Genome Biol., vol. 2, no. 2). In this approach, proteins from two different biological samples are labeled with either Cy3 or Cy5 fluorophores. These two labeled samples are then equally mixed together and applied to an antibody microarray. This approach, for example, allows comparisons to be made between diseased and healthy, or treated and untreated samples. Direct labeling has several advantages, one of which is that the direct-labeling method only requires one specific antibody to perform an assay.

Miniaturized and multiplexed immunoassays may also be used to screen a biological sample for the presence or absence of proteins such as antibodies (Joos et al. 2000, Electrophoresis, vol. 21, no. 13, pp. 2641-2650; Robinson et al. 2002, Nat. Med., vol. 8, no. 3, pp. 295-301).

In a preferred embodiment hereof, the detection or capture agents such as the antibodies are immobilized on a solid support, such as, for example, on a polystyrene surface. In another preferred embodiment, the detection or capture agents are spotted or immobilized in duplicate, triplicate or quadruplicate onto the bottom of one well of a 96-well plate.

In a method hereof, two blood samples will be tested. First, a first sample is tested that has not been exposed to a soluble BCIDT and/or TCIDT. The level of an IFN (preferably type I) response of this sample is determined as a control sample. The level of an IFN (preferably type I) response of this sample is compared to the level of an IFN (preferably type I) bioactivity, IFN (preferably type I)-like bioactivity or IFN (preferably type I) response activity of a second sample from the individual. It is preferred that the first sample and the second sample are of the same tissue.

The second sample has been exposed to BCIDT and/or TCIDT. A sample from an individual who had received the soluble BCID and/or TCID treatment can be used.

Moreover, for response prediction, a cell sample from an untreated individual can be used, wherein the cell sample has been contacted with a soluble BCID or TCID agent in vitro. It is preferred that all samples have been provided with the same soluble BCID or TCID agent.

Accordingly, it is possible to perform a method according to the invention, wherein the at least a second sample has been contacted with soluble BCID or TCID agents in vitro, whereas the first of the samples has been provided with a soluble BCID or TCID agent. This method is also preferred. An advantage thereof is that an in vitro culture with a soluble BCID or TCID agent is technically easier to perform.

In another method, the second sample is exposed in vitro to an IFN (preferably type I) or an inducing agent (preferably type I) such as dsDNA or dsRNA. It is preferred that the first sample and the second sample are of the same tissue.

When a cell sample is used from an individual who had been treated with a soluble BCID or TCID agent, it is preferred to use a sample that is collected at some time after the individual had been exposed to the soluble BCID or TCID agent to allow the soluble BCID or TCID agent to interact with the sample and to allow IFN (preferably type I) genes to respond to the soluble BCID or TCID agent. Preferably, a cell sample is used that is collected between one and four months after the first exposure to the soluble BCID or TCID agent. More preferably, a cell sample that is collected between one and three months after exposure is used, because at the time points, differences between good and poor responders are greater. Expression of genes involved in the IFN (preferably type I) pathway reaches its peak around one to three months after starting a treatment with a soluble BCID or TCID agent. It is preferred that at least two cell samples are collected between one and four months after exposure to a soluble BCID or TCID agent is used, because more samples from different time points increases the accuracy of the method. Most preferably, a cell sample collected at 1, 2, 3 and 4 months after exposure to soluble BCID or TCID agent is used.

When using a cell sample from an individual who did not receive a treatment with the soluble BCID or TCID agent, the cell sample is preferably exposed to a soluble BCID or TCID agent under in vitro conditions. In another method according to the invention the second sample is exposed in vitro to an IFN (preferably type I) or an inducing agent (preferably type I) such as dsDNA or dsRNA.

For in vitro culturing conditions, the cell sample is preferably a blood sample. Preferably, the conditions comprise culturing cells. Culturing procedures for different cell types are well known in the art and a skilled person will be able to select a suitable procedure for the selected cell types.

A method hereof is also suited to prognosticate the clinical response of an individual to the soluble BCID or TCID agent prior to starting a treatment of the individual. To this end, the first sample is tested that has not been exposed to a soluble BCID or TCID agent. An advantage thereof is that such method can be used to determine the prospect of a positive clinical response in individuals before the start of BCIDT and/or TCIDT. The level of IFN (preferably type I) bioactivity, IFN (preferably type I)-like bioactivity or IFN (preferably type I) response activity of this sample from the individual is determined.

Moreover, a method hereof is also suited to prognosticate the clinical response of an individual to the soluble BCID or TCID agent, prior to starting a treatment of the individual if at least two samples are cultured in vitro, in the presence and absence of a soluble BCID or TCID agent. It is understood that if the method is performed using in vitro exposure of a sample, the first and second samples may have been collected as a single sample that is split into a first and second samples. An advantage thereof is that such method can be used to determine the prospect of a positive clinical response in individuals before the start of BCIDT or TCIDT.

It is preferred that the soluble BCID or TCID agent is allowed to interact with the cells and to allow genes involved in the IFN (preferably type I) pathway to respond to the soluble BCID or TCID agent before measuring expression levels of the genes. Preferably, a preferred moment for measuring the expression levels is when the response of the genes is at its peak. A skilled person will be able to establish the most suitable moment to do this by culturing a cell sample taken from an individual prior to therapy with a soluble BCID or TCID agent. Preferably, culturing conditions comprise culturing in the presence of a soluble BCID or TCID agent for 24 to 48 hours. Preferably, the samples comprise blood cells. Preferably, the sample comprises whole blood.

In another method hereof, the second sample is exposed in vitro to an IFN (preferably type I) or an inducing agent (preferably type I) such as dsDNA or dsRNA. It is preferred that the first sample and the second sample are of the same tissue. It is preferred that the IFN (preferably type I) or an inducing agent (preferably type I) such as dsDNA or dsRNA is allowed to interact with the cells and to allow genes involved in the IFN (preferably type I) pathway to respond to the IFN (preferably type I) or an inducing agent (preferably type I) such as dsDNA or dsRNA before measuring expression levels of the genes. Preferably, a preferred moment for measuring the expression levels is when the response of the genes is at its peak. A skilled person will be able to establish the most suitable moment to do this by culturing a cell sample taken from an individual prior to therapy with a soluble BCID or TCID agent. Preferably, culturing conditions comprise culturing in the presence of the IFN (preferably type I) or an inducing agent (preferably type I) such as dsDNA or dsRNA for 24 to 48 hours. Preferably, the samples comprise blood cells. Preferably, the sample comprises whole blood.

Our research findings mark a pharmacological mechanism of action that relies on the induction of changes in the type I IFN response gene activity, which mark concomitant presence of agonistic IFN proteins and/or IFN-inducing agents, upon B-cell depletion by rituximab. B-cells that are targeted via anti-CD20, e.g., rituximab (an anti-CD20 antibody), are rapidly depleted from the peripheral blood CD20-positive B-cells via complement-mediated and antibody-dependent cell-mediated cytotoxicity (ADCC), induction of apoptosis and inhibition of cell growth (D. G. Maloney et al., Rituximab: Mechanism of action and resistance, Semin. Oncol. 2002, 29:2-9). The subsequent release from apoptotic/necrotic material from depleted cells is likely to promote IFN production and release, via TLR-mediated cell activation (S. Akira, S. Uematsu, O. Takeuchi, 2006, Pathogen recognition and innate immunity, Cell 124:783-801). The released IFN on its turn is then responsible for the relatively high increase in IFN (preferably type I) response activity in patients who exhibit relative low IFN (preferably type I) response activity prior to therapy.

These results assigned the IFN pathway as an important pathway that determines the clinical responder status of targeted B-cell depletion therapy. Moreover, the IFN (preferably type I) pathway may underly the pharmacological mechanism underlying the clinical response of rituximab and B- and T-cell depletion, in general. Consequently, this mechanism of action may apply for any cell depletion therapy. Knowing the divergent effects of IFN in disease pathogenesis, the concomitant activation of IFN (preferably type I) may not always be beneficial. The IFN induction in RA is shown to be associated with a beneficial response. A similar mechanism will apply for a disease like MS, where IFNb is known to be beneficial in a subset of the patients. However, the concomitant release of IFN (preferably type I) bioactivity, IFN (preferably type I)-like bioactivity and/or IFN (preferably type I) response activity may have detrimental effects in diseases such as SLE, wherein IFN (preferably type I) contributes to disease pathogenesis, disease activity and/or disease severity.

This disclosure relates to diseases where IFNs, preferably type I IFNs like IFNα, IFNβ or comparable ligands, contribute to disease pathogenesis, disease manifestations and/or severity such as SLE. In a method according to this invention, such patients will have a decreased prospect of a positive clinical or even negative effect on clinical response to a treatment with a. soluble BCID or TCID agent if levels of IFN (preferably type I) bioactivity, IFN (preferably type I)-like bioactivity and/or the expression products of IFN response genes of a treatment are relatively low prior to the start of treatment and/or increase during therapy or in an in vitro-exposed second sample compared to the levels of the same expression products of the first sample. An increased prospect of a poor or negative clinical response to a treatment with soluble BCID or TCID agent in an IFN (preferably type I)-driven disease is thus associated with an absent or relatively low level of IFN (preferably type I) bioactivity, IFN (preferably type I)-like bioactivity and/or expression of IFN response genes in the first sample taken prior to the start of therapy, and/or relatively high increased levels of IFN (preferably type I) bioactivity, IFN (preferably type I)-like bioactivity and/or IFN response activity after the start of therapy with a soluble BCID or TCID agent, compared to levels of the same products of the first sample.

Preferably, the IFN response from the patient sample is compared to a reference. A high IFN response in the sample as compared to a reference indicates the likelihood of a good response to a BCID or TCID. In order to predict a response, the IFN response is preferably significantly different from the reference. The reference may be a reference value, for example, obtained from a different individual or preferably a population, more preferably not afflicted with a disease, wherein IFN contributes to the disease. The reference may also be the level of an IFN response in a second sample from the patient that has been exposed either to a BCID or TCID, or to an IFN or IFN-inducing agent. An expression level is classified as increased at baseline, i.e., prior to the start of therapy with a soluble BCID or TCID agent, when the expression level of the expression product of the first sample is statistically significantly increased in the individual compared to the level of the same expression product found in a sample of a healthy control individual.

The term “significantly” or “statistically significant” refers to statistical significance and generally means a two standard deviation (SD) above normal, or higher, or below, or lower concentration of the expression product. In preferred embodiments, the difference is classified as statistically significant if the expression level is at least a 20 percent increase compared to expression level of the same expression product in control individuals. Preferably, the increase or decrease is at least 20, 25, 30, 35, 40, 45, 50, 75, 100, 150, 200 or 250 percent. Most preferably, the increase or decrease is at least 100 percent.

An expression level is also classified as different when the expression level of the expression product of the second sample is statistically significantly increased or decreased in the individual compared to the level of the same expression product found in the first sample. The term “significantly” or “statistically significant” refers to statistical significance and generally means a two standard deviation (SD) above normal, or higher, or below, or lower concentration of the expression product. In preferred embodiments, the difference is classified as statistically significant if the expression level is at least a 20 percent increased or decreased compared to expression level of the same expression product in control individuals. Preferably, the increase or decrease is at least 20, 25, 30, 35, 40, 45, 50, 75, 100, 150, 200 or 250 percent. Most preferably, the increase or decrease is at least 100 percent.

More preferred is a method, wherein the IFN (preferably type I) response level is determined by determining in the first sample or at least two samples the level of an expression product of at least one gene of Table 2. An advantage thereof is that these genes are more predictive. More preferably, 2, 3, 4, 5, 6, 7, 8, 9, 10, 11, 12, 13, 14, 15, 20, 25, 30 or 34 genes of Table 2 are used, because the inclusion of more genes of this table improves the accuracy of the method.

More preferred is a method wherein one level of the IFN (preferably type I) response is determined by determining the level of an expression product of at least EPST1, HERC5, LY6E, ISG15, Mx1, OAS1, LGALS3BP, RSAD2, IFI44, IFI44L, MX2, OAS2, BAFF, DARC and/or SERPING1. An advantage thereof is that these genes have a good predictive power.

In a preferred embodiment hereof, the IFN (preferably type I) response is determined by determining the level of an expression product of at least RSAD2, HERC5, ISG15, IFI44L, Ly6E and Mx1. An advantage thereof is that the use of these genes results in a good predictive power.

In another preferred embodiment hereof, the IFN (preferably type I) response is determined by determining the level of an expression product of at least RSAD2, HERC5, ISG15, IFI44L, Ly6E and Mx1. These genes have a good predictive power.

Even more preferred is an embodiment wherein the IFN (preferably type I) response is determined by further determining the level of an expression product of at least RSAD2, HERC5, ISG15, IFI44L, Ly6E and Mx1. An advantage thereof is that the combined use leads to an improved predictive power.

Another preferred embodiment is a method wherein the IFN (preferably type I) response is determined by determining the level of an expression product of at least EPSTI1, LGALS3BP, RSAD2, HERC5, ISG15, IFI44L, Ly6E, Mx1, OAS1, and IFI44. More preferably, the IFN (preferably type I) response is determined by determining the level of an expression product of at least EPSTI1, LGALS3BP, RSAD2, HERC5, ISG15, IFI44L, Ly6E, Mx1, OAS1, and IFI44 and SERPING1.

More preferably, the IFN (preferably type I) response is determined by determining the level of an expression product of at least the 15 validation genes listed in Tables 1 and 2 (including BAFF and DARC). This further improves the predictive power of the method. Most preferred is a method wherein at least the 34 genes listed are used (C. T. M. Van der Pouw Kraan, et al., Rheumatoid arthritis subtypes identified by genomic profiling of peripheral blood cells: assignment of a type I interferon signature in a subpopulation of patients, Annals of Rheumatic Dis. 2007, 66:1008-1014).

In another aspect, the disclosure relates to a method for prognosticating a clinical response of a patient to a treatment with a soluble BCID or TCID agent, the method comprising determining the level of the expression products of the genes listed in Tables 1 and 2 (including BAFF and DARC) in the first sample prior to the start of therapy with soluble BCID or TCID agent, or at least two samples of the individual, wherein a first of the samples has not been exposed to a soluble BCID or TCID agent and wherein at least a second of the samples has been exposed to a soluble BCID or TCID agent prior to determining the level, the method further comprising comparing the levels and prognosticating the clinical response from the comparison.

More preferred is a method wherein the at least one gene comprises 2, 3, 4, 5, 6, 7, 8, 9, 10, 11, 12, 13, 14, 15, 16, 17, 18, 19, 20 or 21 genes of Tables 1 and 2 (including BAFF and DARC).

More preferred is a method wherein the at least two samples comprise cell samples. An advantage thereof is that cells samples comprise nucleic acids, which can advantageously be used for determining the levels of an IFN (preferably type I) response, the level of expression product of the genes and/or the at least one gene listed in Tables 1 and 2 (including BAFF and DARC).

More preferred is a method wherein the second sample is of an individual between one and four months after the first exposure of the individual to the soluble BCID or TCID agent. An advantage thereof is that within this period, the IFN (preferably type I) response level or the level of the expression products of Tables 1 and 2 differs significantly compared to the first sample.

In another aspect, the disclosure relates to a method for treatment of a patient with a soluble BCID or TCID agent, comprising determining a prognosis for a clinical response to a treatment with the soluble BCID or TCID agent, further comprising treating the individual with the soluble BCID or TCID agent, if the individual has been prognosticated as a good responder.

In another aspect, the disclosure relates to use of a soluble BCID or TCID agent for the preparation of a medicament for the treatment of a patient, wherein prior to the treatment, a prognosis for a clinical response to the soluble BCID or TCID agent was determined with any of the methods described above.

In another aspect, the disclosure relates to an improved pharmacodynamic marker (PD marker) for evaluating a pharmacological effect of a treatment with a soluble BCID or TCID agent. Good PD markers are needed to improve the prediction of the efficacy and safety of a treatment with a soluble BCID or TCID agent at the individual patient level. These quantitative PD markers should reflect features of drug exposure and drug response with respect to modulation of the molecular target, the cognate biochemical pathways and/or downstream biological effects. The availability of quantitative PD markers provides a rational basis for decision making during, e.g., treatment optimization. A PD marker currently described for rituximab is peripheral blood B-cell levels. Various reasons for inadequate depletion have been proposed, including genetic polymorphisms of the FcRyIIIa gene (J. H. Anolik et al., The relationship of FCyRIIIa genotype to the degree of B-cell depletion by rituximab in the treatment for systemic lupus erythematosus, Arthritis Rheum. 2003, 48:455-459) or defective complement. Other PD markers include levels of autoantibodies that are described to be down-regulated in patients treated with soluble BCID or TCID agent who show a clinical response (G. Cambridge et al., Serologic changes following B-lymphocyte depletion therapy for rheumatoid arthritis, Arthritis Rheum. 2003, 48:2146-2154). However, neither fully explains the response status. Moreover, most of the described PD markers are assessed by using mean levels of patient groups while most of these markers are not affected in each individual patient.

Provided is a method for evaluating a pharmacological effect of a treatment of a patient with a soluble BCID or TCID agent, the method comprising determining the level of an expression product of at least one gene of Table 2 in at least two samples of the individual, wherein a first of the samples has not been exposed to a soluble BCID or TCID agent and, wherein at least a second of the samples has been exposed to the soluble BCID or TCID agent prior to determining the level. The method is preferably used to determine whether the moment of renewed therapy and dose of a soluble BCID or TCID agent that a patient receives is well timed and sufficiently high to achieve an effect or a clinical response. Whether a clinical response can be achieved depends also on other factors.

The method can also be used to determine whether the dose of a soluble BCID or TCID agent that a patient receives is not too high and might, therefore, cause side effects. With the term “pharmacological effect” is meant a biochemical or physiological effect of a soluble BCID or TCID agent. Preferably, such pharmacological effect is specific for a treatment with a soluble BCID or TCID agent. Preferably, such pharmacological effect reflects the relationship between an effective dose and the clinical response. Preferably, the effective dose is the dose as measured in the blood level. With the term “evaluating” is meant that results of a pharmacological effect is determined and used for decision-making steps regarding further treatment. Preferably, “evaluating” comprises evaluating the dose, the efficacy and/or the safety of the soluble BCID or TCID agent. Preferably, the expression products of at least 2, 3, 4, 5, 6, 7, 8, 9, 10, 15, 25, genes of Tables 1 and 2 (including BAFF and DARC) are used in the method. Other terms used are explained above. Preferably, the expression products of the genes comprise the genes, wherein the level of the expression product is higher than 1.5, 1.6, 1.7, 1.8, 1.9, 2.0, 2.1, 2.2, 2.4, 2.6 or 3.0, or lower than 0.68, 0.67, 0.66, 0.65, 0.64, 0.62 or 0.60 (see column “fold change,” Tables 1 and 2, including BAFF and DARC). Values equal or somewhat increased than T=3 month values and in “fold change” and reaching baseline expression values in Tables 1 and 2, including BAFF and increased DARC, are indicative of renewed administration and eventually increased dose of a soluble BCID or TCID agent for disease that benefit from an increased IFN response activity like RA and MS. Likewise, the opposite protocol applies for diseases, such as SLE, wherein IFNs (preferably type I) and/or IFN (preferably type I) response activity contributes to disease severity and/or activity. Values higher than 1 in “fold change” and resembling T3 values as listed in Tables 1 and 2 and BAFF and DARC are indicative of a prolonged renewal of treatment. Up-regulation of a gene having a “fold change” higher than 1 as listed in Table 2 is indicative of an effective dose of a soluble BCID or TCID agent in diseases that benefit from IFN (preferably type I) activity.

As mentioned above, a moderate increase may be tolerated for patients with diseases like SLE, wherein IFN (preferably type I) activity is contributing to disease severity and activity, whereas a high increase is not preferred and reason to lower the dose or stop therapy. In another preferred embodiment, the at least one gene preferably comprises genes and gene products that are responsive to IFN (preferably type I). See Tables 1 and 2 for further details on the mentioned genes. Preferred is a method wherein at least the second of the samples has been exposed to a soluble BCID or TCID agent prior to determining the level.

Another preferred embodiment is a method wherein at least the second of the samples has been exposed to a soluble BCID or TCID agent prior to determining the level.

An advantage of this method is that the level of at least one gene of Tables 1 and 2 (including BAFF and DARC) reflects a good clinical response to a therapy with a soluble BCID or TCID agent. Therefore, the response reflects drug activity and can be used to monitor drug efficacy at the individual patient level. Drug efficacy is the ability of a drug to produce the desired therapeutic effect.

In another aspect, the disclosure relates to a method for treatment of a patient with a soluble BCID or TCID agent, wherein the dose of the soluble BCID or TCID agent treatment is based on results obtained by a method for evaluating a pharmacological effect of a treatment of a patient with a soluble BCID or TCID agent, the method comprising determining the level of a pharmacogenomic response of at least one gene of Tables 1 and 2 (including BAFF and DARC) in at least two samples of the individual, wherein a first of the samples has not been exposed to a soluble BCID or TCID agent and wherein at least a second of the samples has been exposed to a soluble BCID or TCID agent prior to determining the level. The term “based on” means that results of the method are taken into account in establishing the dose of the soluble BCID or TCID agent most suited for the individual patient. Preferred is a method wherein a patient is treated with a soluble BCID or TCID agent and wherein the method for evaluating a pharmacological response is based on results obtained by a method for evaluating a pharmacological effect, wherein the at least a second of the samples has been exposed to a soluble BCID or TCID agent prior to determining the level.

In another aspect, the disclosure relates to use of a soluble BCID or TCID agent for the preparation of a medicament for the treatment of a patient, wherein the treatment is evaluated based on a method for evaluating a pharmacological effect of a treatment of a patient with a soluble BCID or TCID agent, the method comprising determining the level of a pharmacogenomic response of at least one gene of Tables 1 and 2 (including BAFF and DARC) in at least two samples of the individual, wherein a first of the samples has not been exposed to a soluble BCID or TCID agent and wherein at least a second of the samples has been exposed to the soluble BCID or TCID agent prior to determining the level. Another preferred embodiment is the use of a soluble BCID or TCID agent for the preparation of a medicament for the treatment of a patient and wherein the method for evaluating a pharmacological response is based on results obtained by a method for evaluating a pharmacological effect, wherein the at least a second of the samples has been exposed to a soluble BCID or TCID agent prior to determining the level.

In another aspect, the disclosure relates to a method for treatment of a patient with a soluble BCID or TCID agent, wherein the prediction of the response to BCID or TCID, pharmacological monitoring and dosing of the soluble BCID or TCID agent is based on results obtained by a method for evaluating the second sample that is exposed in vitro to an IFN (preferably type I) or an inducing agent (preferably type I) such as dsDNA or dsRNA. It is preferred that the first sample and the second sample are of the same tissue. It is preferred that the IFN (preferably type I) or an inducing agent (preferably type I) such as dsDNA or dsRNA is allowed to interact with the cells and to allow genes involved in the IFN (preferably type I) pathway to respond to the IFN (preferably type I) or an inducing agent (preferably type I) such as dsDNA or dsRNA before measuring expression levels of the genes. Preferably, a preferred moment for measuring the expression levels is when the response of the genes is at its peak. A skilled person will be able to establish the most suitable moment to do this by culturing a cell sample taken from an individual prior to therapy with a soluble BCID or TCID agent. Preferably, culturing conditions comprise culturing in the presence of the IFN (preferably type I) or an inducing agent (preferably type I) such as dsDNA or dsRNA for 24 to 48 hours. Preferably, the samples comprise blood cells. Preferably, the sample comprises whole blood. An advantage thereof is that within this period, the IFN (preferably type I) response level or the level of the expression products of Tables 1 and 2 differs significantly compared to the first sample. An advantage thereof is that cell samples comprise nucleic acids, which can advantageously be used for determining the levels of an IFN (preferably type I) response, the level of expression product of the genes and/or the at least one gene listed in Tables 1 and 2 (including BAFF and DARC). Preferably, the expression products of at least 2, 3, 4, 5, 6, 7, 8, 9, 10, 15, 25, genes of Tables 1 and 2 (including BAFF and DARC) are used in the method. Other terms used are explained above. Preferably, the expression products of the genes comprise the genes, wherein the level of the expression product is higher than 1.5, 1.6, 1.7, 1.8, 1.9, 2.0, 2.1, 2.2, 2.4, 2.6 or 3.0, or lower than 0.68, 0.67, 0.66, 0.65, 0.64, 0.62 or 0.60 (see column “fold change,” Tables 1 and 2 including BAFF and DARC).

In another aspect, the disclosure relates to a kit suitable for use in the above method, comprising up to 34 reagents, sequence-specific oligonucleotides and/or capture agents for detecting up to 34 of the gene products listed (C. T. M. Van der Pouw Kraan et al., Rheumatoid arthritis subtypes identified by genomic profiling of peripheral blood cells: assignment of a type I interferon signature in a subpopulation of patients, Annals of Rheumatic Dis. 2007, 66:1008-1014) and of the genes listed in Tables 1 and 2.

In another aspect, the disclosure relates to a kit suitable for use in the above method, comprising up to Tables 1 and 2 and including BAFF and DARC.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C: Relation between IFN signature at baseline and decrease in DAS28 score at six months.

FIG. 1A. Patients (n=12) were separated in EULAR good, moderate and poor responders, and into ΔDAS28<1.2 and ΔDAS28>1.2 groups. Subsequently, the association with baseline IFN gene expression activity (mean expression of set of 12 type I IFN genes) (y-axis) was determined (T-test; P=0.0395).

FIG. 1B. Cluster diagram showing the genes that discriminate between ΔDAS28 responders and non-responders (the most informative ones are given in the window).

FIG. 1C. Cluster of genes whose increased expression levels at baseline (represented in red) is associated with a good clinical response.

FIG. 2: Cluster diagrams of genes that were differentially regulated by rituximab between RA patients. Panel A, unsupervised (two-way) hierarchical cluster analysis of induced gene expression levels (ratio t3/t0) of a set of 154 genes revealed a marked inter-individual variation in the pharmacological response to rituximab between RA patients. A total of six clusters of genes that were differently regulated at three months following the start of rituximab therapy between patients. Pathway level analysis revealed that the clusters contained genes related to type I IFN biology (cluster A), Protein translation, MIF- and TCR-signaling and NK-cell cytotoxicity (cluster B), B-cell immunology (cluster C), ECM modeling and connective tissue degradation (cluster D), chemotaxis, adhesion and S100 family proteins (cluster E). Cluster F consisted of many genes with unknown function that together could not be classified into a pathway. Panel B, supervised (one-way) cluster analysis revealed a set of type I IFN-response genes associated with clinical outcome. Patients were stratified based on changes in Disease Activity Score (ΔDAS) at six months after the start of therapy. Panel C, cluster of type I IFN response genes, which is related to clinical responder status.

FIG. 3: Differential regulation of type I IFN response genes upon rituximab therapy. The expression levels of six type I IFN response genes were determined by cDNA-microarray analysis in peripheral blood cells of 13 RA patients before (t=0) and three months after (t=3) rituximab treatment. For each patient, the expression levels were averaged (in log2 space) and the induction (ratio t=0/t=3) calculated. Data are shown as box plots; each box showed the 25^{th} to 75^{th} percentiles. Box Plot A, T-test analysis revealed a significant increase in the expression of the type I IFN response genes in responders compared to non-responders based on ΔDAS> or <1.2. Box Plot B, patients were divided into two groups based on changes in gene expression levels of the type I IFN response gene set (ratio< or >0.15). The groups were compared to each other with respect to ΔDAS28 improvement. This cut-off point marked a significant classification between clinical response status of the patients. Box Plot C, the expression levels RSAD2 was determined by qPCR in peripheral blood cells of an independent validation cohort of nine RA patients before (t=0) and three months after (t=3) rituximab treatment. For each patient, the induction (ratio t=0/t=3, log2 space) was calculated. MannWhitney U test analysis revealed a significant increase in the expression of RSAD2 in responders compared to non-responders based on ΔDAS> or <1.2.

FIG. 4: Dynamics of the CD20 B-cell counts and type I IFN response signature during rituximab treatment. Expression levels of type I IFN-related gene expression level (left y-axis) and B-cell counts (right y-axis) at baseline, three (t=3) and six months (t=6) in responders and non-responders based on ΔDAS (Panel A) and EULAR (Panel B) criteria are shown. Baseline type I IFN response gene expression levels were significantly different between good responders (ΔDAS>1.2 or EULAR) and non-responders (ΔDAS<1.2 or EULAR) (p=0.0052 and p=0.048, respectively). In both groups, gene expression levels return to baseline values six months post therapy. No differences in B-cell count between groups are observed.

### DETAILED DESCRIPTION

### Materials and Methods

### Patients

Consecutive patients with RA according to the ACR criteria were enrolled in the study. Inclusion criteria were: 18-85 years of age, a failure of at least two disease-modifying anti-rheumatic drugs (DMARDs) including methotrexate (MTX), and active disease (DAS28≧3.2). Patients who failed on previous use of a TNF-blocking agent were included. Patients were on stable maximally tolerable MTX treatment. Whole blood samples (2.5 ml) were obtained using PAXgene tubes (PreAnalytix, GmbH, Germany) from 15 RA patients prior to initiation of therapy with rituximab (3 mg/kg intravenously at baseline, after x weeks). After three and six months of treatment, another PAXgene tube was obtained. All patients gave written informed consent and the study protocol was approved by the Medical Ethics Committee. After 24 weeks of treatment, the clinical response to treatment was assessed using both the EULAR criteria as well as the reduction in DAS28 of at least 1.2.

### Treatment and Clinical Evaluation

Patients received rituximab 1,000 mg intravenously on days 1 and 15, in combination with clemastine (2 mg intravenously), methylprednisolone (100 mg intravenously) and acetaminophen 1,000 mg orally, as premedication. Every four weeks after the first infusion and from 12 weeks on, every three months, patients were assessed for disease activity by the 28 joints Disease Activity Score (DAS28)^{[12]} and blood sampling. The use of concomitant DMARDs, prednisolone or non-steroidal anti-inflammatory drugs (NSAIDs) during the study duration was permitted. Response to treatment was classified according to EULAR response and to change to DAS28.^{[12]}

### Blood Sampling and RNA Isolation

For RNA isolation, 2.5 ml blood was drawn in PAXgene tubes (PreAnalytix, GmbH, Germany) and stored at −20° C. Tubes were thawed overnight at room temperature prior to RNA isolation. Total RNA was isolated using Bio robot MDX (Qiagen, Benelux b.v., Venlo, The Netherlands) according to the manufacturer's instructions (PAXgene Blood RNA Mdx kit). Samples were cleaned from salts that may be present using Qiagen RNA MinElute procedure according to the manufacturer's procedure (Qiagen, Venlo, The Netherlands). Total RNA concentration was measured using the Nanodrop spectrophotometer (Nanodrop Technologies, Wilmington, Del.) and RNA purity and integrity was verified using lab-on-chip technology (Agilent 2100 Bioanalyzer, California, USA).

### Microarray Analysis

The ILLUMINA® TOTALPREP™ RNA amplification kit (Ambion, Austin, Tex., USA) was used to synthesize biotin-labeled cRNA from 500 ng total RNA. Concentration of the labeled cRNA was measured using Nanodrop spectrophotometer and 750 ng biotinylated cRNA was hybridized onto the HumanHT-12 v3 Expression BeadChip (Illumina, San Diego, Calif.).

Amplification and hybridization were performed at the outsourcing company ServiceXS (Leiden, the Netherlands). Bead summary intensities were log2-transformed and normalized using quantile normalization.^{[13, 14]}

cDNA Synthesis and Quantitative Real Time PCR

RNA (0.5 μg) was reverse transcribed into cDNA using a Revertaid H-minus cDNA synthesis kit (MBI Fermentas, St. Leon-Rot, Germany) according to the manufacturer's instructions. Quantitative real-time PCR was performed using an ABI Prism 7900HT Sequence detection system (Applied Biosystems, Foster City, Calif., USA). Gene expression levels of two genes (GAPDH, RSAD2) were determined using Taqman Gene expression assays following manufacturer's guidelines. To calculate arbitrary values of mRNA levels and to correct for differences in primer efficiencies for each gene, a standard curve was constructed. Expression levels of target genes were expressed relative to housekeeping gene glyceraldehydes-3-phosphate dehydrogenase (GAPDH).

### Flow Cytometry

In order to determine the relative amount of peripheral T- and B-lymphocytes, whole blood was stained for 30 minutes with fluorescein isothiocyanate (FITC), phycoerythrin (PE), peridinin chlorophyll protein (PerCP) and allophycocyanin (APC) conjugated monoclonal antibodies directed against lymphocyte subset-associated surface molecules. Four color antibody combinations used were (FITC/PE/PerCP/APC): CD3/CD8/CD45/CD4 and CD3/CD16+56/CD45/CD19 (all from BD Biosciences, San Jose, Calif.). Following staining, the red cells were lysed (Lysing Solution, BD Biosciences) and lymphocyte subsets were analyzed by flow cytometry. Flow cytometric analysis was performed on a standard four-color Fluorescence Activated Cell Scanner (FACSCalibur, BD Biosciences). The data were analyzed using Cellquest Pro software (BD Biosciences). Care was taken to analyze only viable cellular events based on light scatter properties. All analyses were performed on lymphocytes, based on bright CD45 staining and low sideward scatter.

### Statistical Analysis

Statistical analysis on microarray data was performed using Significant Analysis of Microarray data (SAM) version 3.09.^{[15]} Two class paired analysis using Statistical Analysis of Microarray data (SAM) at a false discovery rate (FDR) of less than 5% between pre- and post-therapy data was applied to identify genes that were significantly changed in expression after rituximab therapy. Cluster analysis was used for the subclassification of coordinately differentially expressed genes.^{[16]} Treeview was used to visualize differentially expressed genes. Gene Set Enrichment Analysis (GSEA; on the World Wide Web at broad.mit.edu/gsea) was used for pathway analysis.^{[17,18]} We used gene set permutation to adjust for multiple testing, indicated by a false discovery rate. A minimal gene set size of 15 genes per pathway was applied, and pathways with a p-value of <0.05 and a FDR of <0.05 were considered significant. A total of 282 pathways from Biocarta and KEGG were applied in this analysis. In addition, we incorporated the IFN response gene set^{[19]} (genes with at least a five-fold up-regulation in PBMC after treatment with type I IFN).

For ontology analysis of gene sets identified by cluster analysis, we used METACORE™ Pathway analysis, using the METACORE™ Ontology tools, developed by GeneGo (GeneGO, St Joseph, Mich., on the World Wide Web at genego.com/). Data mining in METACORE™ is based on a manually curated database of human protein-protein, protein-DNA interactions, transcription factors, signaling pathways and metabolic pathways. Calculation of statistical significance are based on p-values, which are defined as the probability of a given number of genes from the input list to match a certain number of genes in the functional GeneGO Gene Ontology categories.

Differences in gene expression levels of IFN response genes between patients with a ΔDAS28>1.2 versus ΔDAS28<1.2 or between EULAR good versus moderate versus non-responders were analyzed using Student's unpaired t-test or Mann-Whitney U test, where appropriate.

### Results

### Relationship Between Clinical Response and Baseline IFN Response Gene Activity

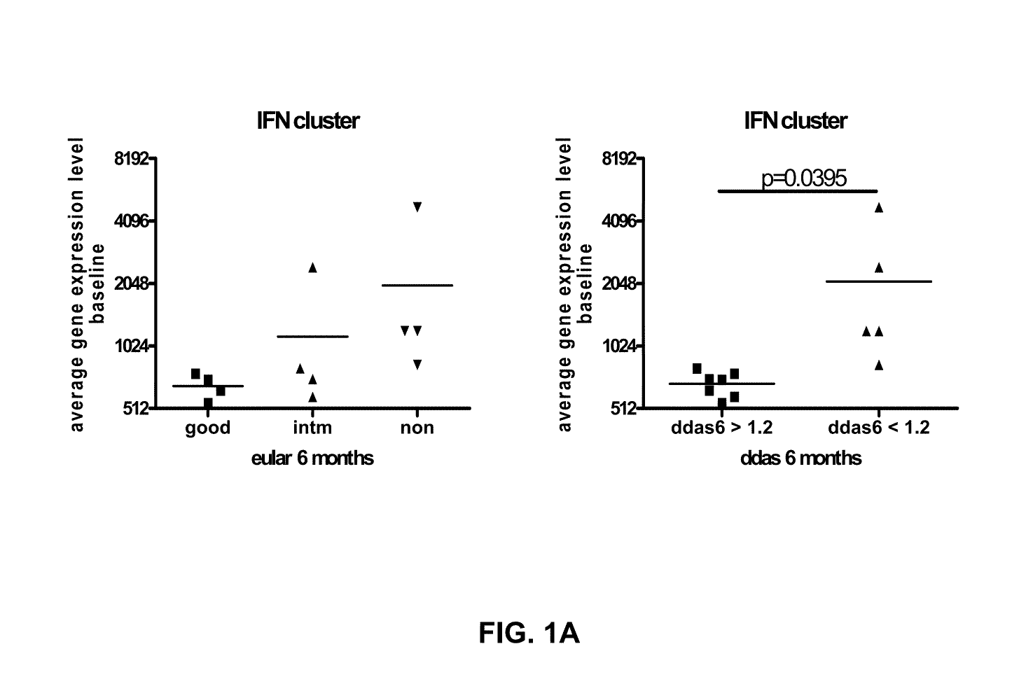

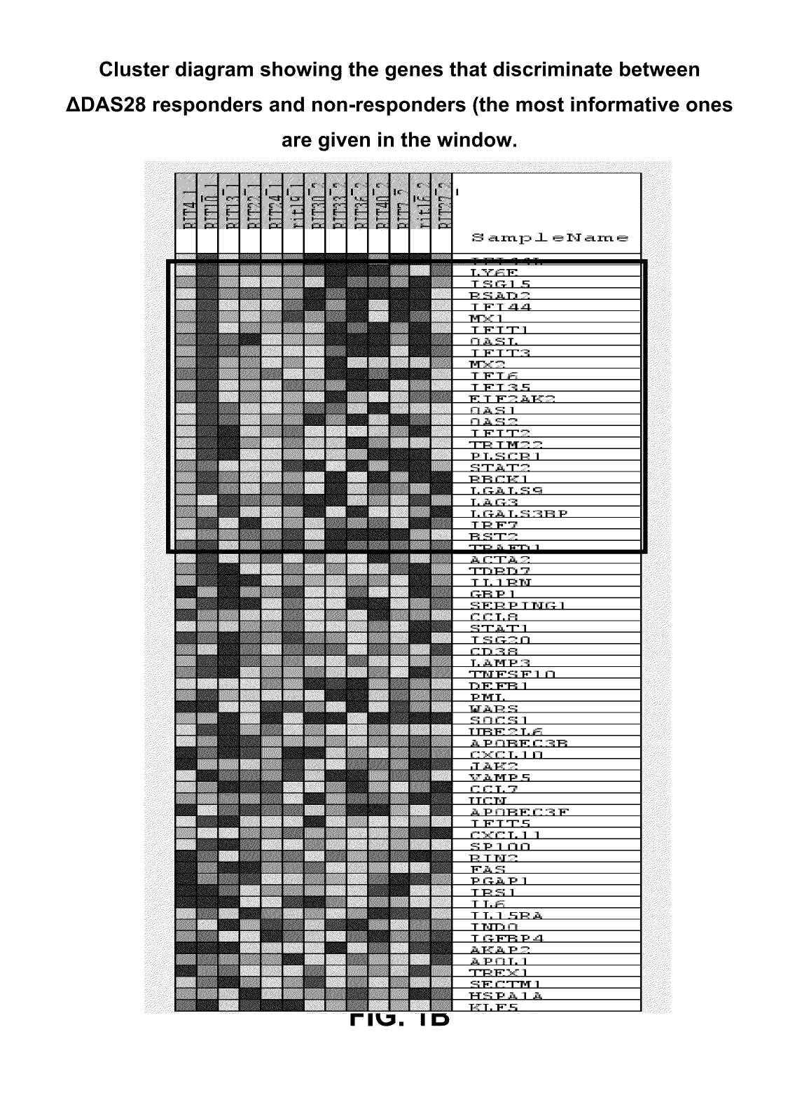

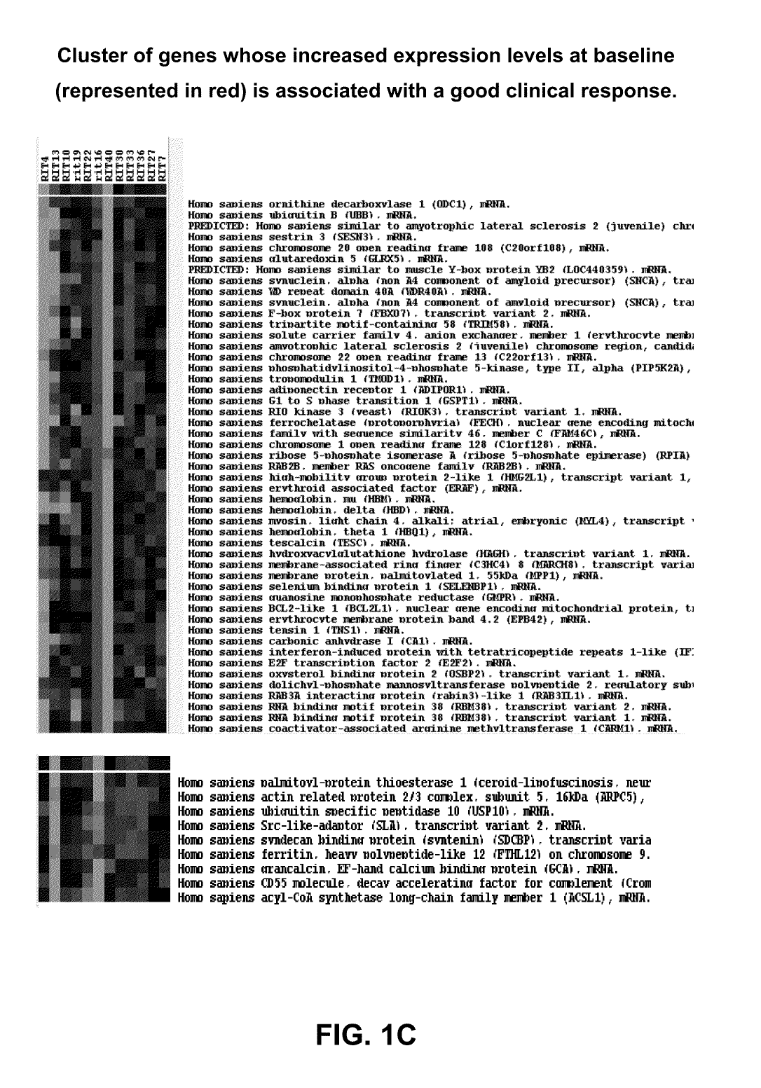

Previously, we demonstrated significant differences in the expression of IFN response genes between biologically naive RA patients. Here, we studied whether baseline IFN response gene activity is associated with clinical response to rituximab. Therefore, we performed genome-wide gene expression profiling on peripheral blood cells from patients with RA before the start of therapy with rituximab. Supervised hierarchical cluster analysis was done using the baseline gene expression profiles. Therefore, patients were stratified on the basis of responders and non-responders based on ΔDAS response criteria. This analysis revealed that type I IFN response gene activity at baseline is significantly increased in the non-responders compared to the non-responders (p=0.0395) (FIG. 1). These findings were confirmed in an independent cohort of 50 patients (p=0.012). Similar findings were based when patients were stratified based on EULAR response criteria. Subsequently, statistical analysis of microarrays and correlation analysis was performed to further compare baseline gene expression levels in patients that were responders versus non-responders (Tables 1A, 1B, 1C and 1D). Among others, in particular, monitoring EPSTI1, HERC5, IFI44L, Mx1, RSAD2, LY6E, ISG15, IFI44, BAFF, DARC, OAS1, LGALS3BP, Mx2, OAS2 and SERPING1 were good discriminators between responders and non-responders. Comparing the average baseline levels of 12 IFN response genes shows significant differences between patients with a ΔDAS28>1.2 and those with a ΔDAS28<1.2 (FIG. 1). Thus, baseline levels of IFN type I response activity in peripheral blood determine the clinical response status to treatment with rituximab.

For a broader identification of gene patterns associated with responder status, supervised clustering was performed, whereby patients were a priori categorized in predetermined groups based on EULAR response criteria. Genes were selected that differed at least two-fold in at least three samples. When analyzing the gene expression clusters that are determined by the categorization of responders and non-responders, we observed a cluster of IFN type I response genes that showed increased expression in the non-responders and a relatively low expression in the responders. Additionally, patients were ranked based on increasing ΔDAS28 response criteria. Also here, hierarchical cluster analysis learned that a good response (ΔDAS28>1.2) is observed for those patients with a low level of expression of type I IFN response genes at baseline. The genes that comprise the IFN response signature and other genes that discriminate between responders and non-responders are listed in Tables 1A, 1B, 1C and 1D. Relevant genes comprising the IFN signature are represented in Tables 1A, 1B, 1C and 1D).

The baseline expression of one of the most pronounced IFN-response genes Mx1 appeared to negatively correlate with the mRNA expression levels of DARC (r=−0.61; P=0.035). Moreover, a positive correlation was observed between Mx1 and the expression of B-cell-activating factor BAFF (r=0.682; P=0.014). In addition, a number of genes at baseline revealed an increased expression that correlated with a good clinical response (ΔDAS28 or EULAR) (FIG. 1C).

In an independent study, the presence of an IFN signature was measured in peripheral blood mononuclear cells from baseline using polymerase chain reaction on the three interferon-regulated genes Mx1, ISG15 and OAS1. After comparison with healthy controls, patients were qualified as IFN high or IFN low. In this cohort (n=50), a significantly lower decrease in DAS28 was observed in the IFN high patients (n=24) at week 24 compared to the IFN low group (n=26; mean (±SD)-0.90 (+1.5) compared to −2.0 (±1.4); P=0.012). Accordingly, less patients obtained a EULAR response in the IFN high group compared to the IFN low group when the data are pooled (P=0.032).

Moreover, higher levels of the following cytokines were measured in the IFN high group (P<0.01): IL1β, IL4, IL12, IL13, IL18, IL21, IL23, IFNγ, MIP3β, and hyaluronzuur (synovial injury marker) (p=0.005). Also, certain cell surface markers, such as Sialic acid-binding Ig-like lectin 1 (Siglec-1, sialoadhesin, CD169, CD64), are known as prominent type I IFN-regulated candidate genes. (Biesen et al., Arthr. Rheum. 2008 April, 58(4):1136-45.) We claim the use of markers such as Siglec and/or CD64 as a marker for IFN activity in RA and for the use of predicting and monitoring therapy response with biological.

Altogether, the data reveal that the increased presence of the type I IFN signature before the start of therapy negatively predicts the clinical response to rituximab treatment in RA. These data support the notion that type I IFN signaling plays a role in RA immunopathology.

### Pharmacological Response to Rituximab Treatment in RA

In order to determine the pharmacological effects of rituximab, we analyzed the changes in peripheral blood whole genome expression profiles of 13 RA patients at baseline, and three and six months following the start of treatment. To search for single genes that were significantly regulated in all patients after three months of treatment with rituximab, we applied two class paired analysis using Statistical Analysis of Microarrays (SAM) at a False Discovery Rate (FDR) of less than 5% between pre- and post-therapy. The analysis revealed 16 genes that were significantly down-regulated in all patients. As anticipated, these genes were B-cell-related genes confirming observations by others of an effective and selective B-cell depletion after three months of therapy. All patients reached a comparable low level of expression of B-cell-related genes, indicative that the pharmacological depletion at three months was reached irrespective of the clinical response status. Accordingly, pathway level analysis using Gene Set Enrichment Analysis (GSEA), identified “B-cell-mediated immunity” as the only significantly down-regulated pathway (p=0.0020). At six months following therapy, only six of the B-cell-related genes were still significantly down-regulated. At both time points, no genes were significantly up-regulated. These data are indicative for a gradual rise in B-cell markers from three to six months following the start of therapy. These findings were confirmed by CD19-based FACS analysis (data not shown). Altogether, this indicates that under the influence of rituximab, only changes in B-cell-related processes were consistently regulated in all patients.

### Variation in the Pharmacological Response to Rituximab Between RA Patients

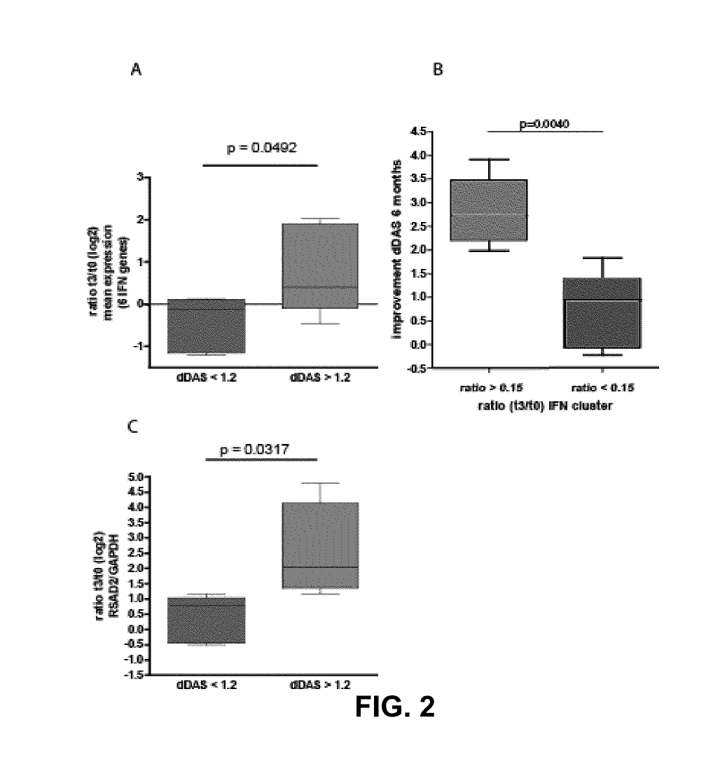

Given the heterogeneous nature of RA and the relatively low number of differentially regulated genes in the group-based analysis, we questioned how consistent the pharmacological response to rituximab was between RA patients. Therefore, we analyzed the pharmacological effects of each individual patient by comparison of the ratio of the post- (three months) vs pre-therapy expression level for each gene (log-2 ratios). To search for differences in the pharmacological response between patients, 154 genes were identified that revealed at least a two-fold difference in the rituximab-induced response in at least three patients (FIG. 2). Altogether, these analyses show that pharmacological responses in RA patients under the influence of rituximab treatment are highly heterogeneous between patients.

### Pharmacodynamics in Relation to Clinical Response

Next, we investigated the pharmacological differences between patients in relation to clinical response. Therefore, patients were stratified based on changes in Disease Activity Score (ΔDAS) at six months after the start of therapy in good responders (ΔDAS>1.2; n=7) and non-responders (ΔDAS<1.2; n=6) (FIG. 2). Subsequently, we performed a cluster analysis using the set of 154 genes to search for genes that were differentially regulated by rituximab between responders and non-responders. Remarkably, the analysis revealed that only a selective increase in the expression of type I IFN response genes at three months following the start of rituximab therapy correlated with a good clinical outcome. Those patients who had a similar or decreased expression of type I IFN genes exhibited a poor response. This association was most prominent for genes that constitute a subcluster of six genes consisting of IFI44, IFI44L, HERC5, RSAD2, LY6E and Mx1, which were used for further analyses (FIG. 2, Panel C). None of the other differentially regulated gene clusters were associated with clinical responsiveness.

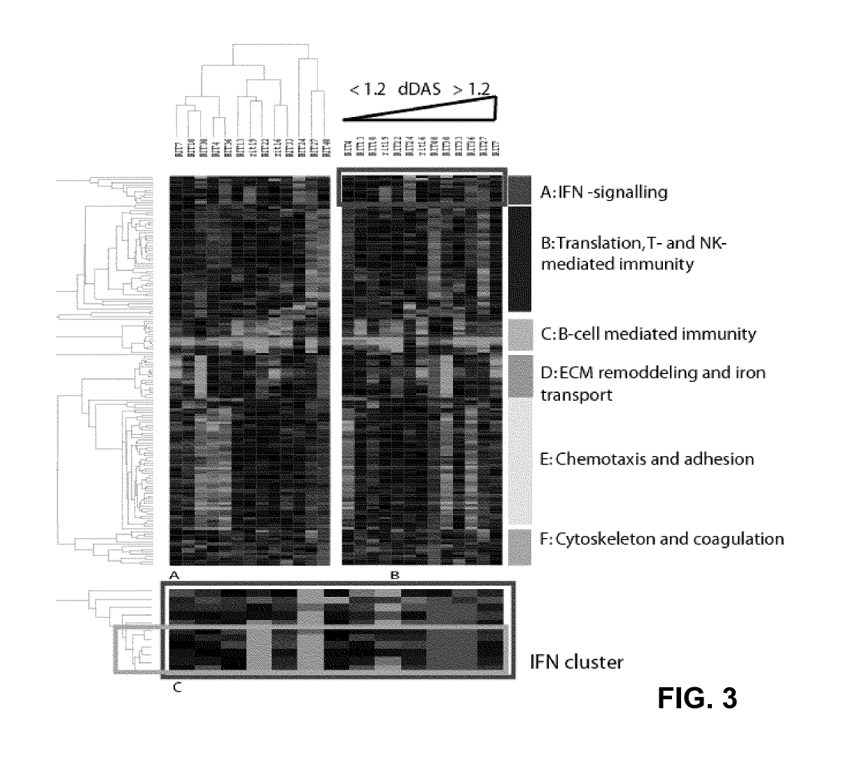

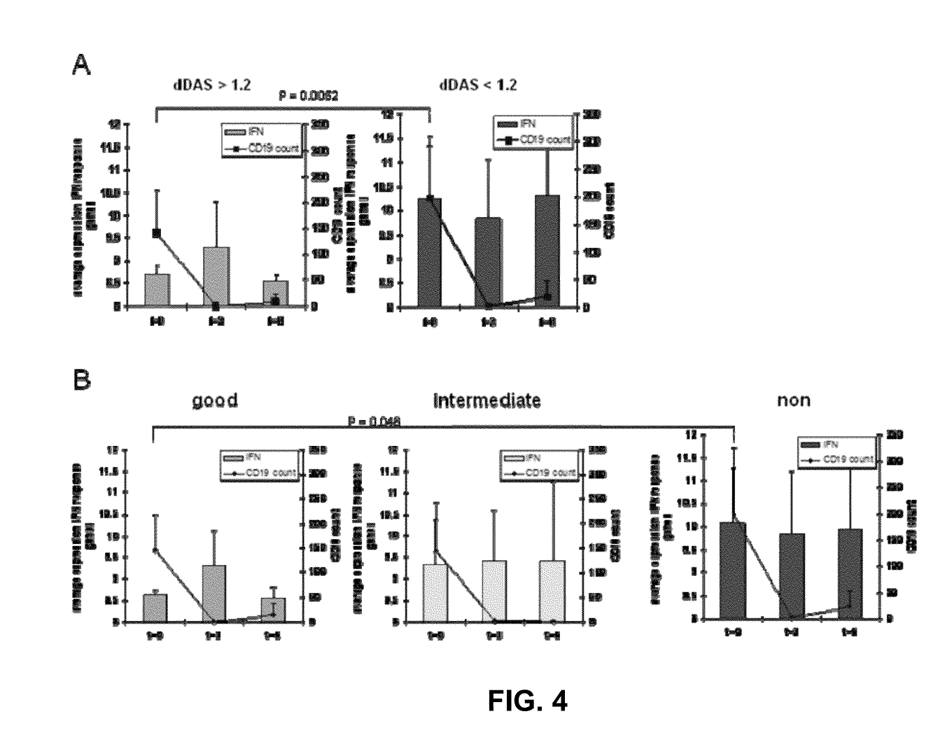

For further analysis, the expression levels of these six type I IFN response genes at each time point were averaged to reach an IFN type I response score for each individual patient. Treatment induced changes over the three-month time period were compared between the responders (ΔDAS>1.2) and non-responders (ΔDAS<1.2) using a student's t-test. This analysis revealed a significant increase in the IFN response score in the responders compared to the non-responders (p=0.0492, FIG. 3). Moreover, we observed that division of patients into two groups based on a cut-off of 0.15-fold (log2 based) induction of this gene set resulted in a clear classification of good responders (high ΔDAS) and non-responders (low ΔDAS) (p=0.0040, FIG. 3). Accordingly, similar results were observed when response status was assessed by the EULAR response criteria in good responders (n=4), intermediate responders (n=4) and non-responders (n=5) (p=0.048, data not shown). The expression of the IFN response gene set returned to baseline values at six months after the start of therapy (FIG. 4).

To confirm the increase in type I IFN response activity at three months following the start of rituximab therapy, we used an independent cohort of nine patients (four good and five non-responders based on ΔDAS), and measured the expression of RSAD2, a representative IFN type I response gene that has a high correlation with the mean expression value of the IFN type I response gene set, at baseline and three months. This analysis validated the findings of a significant increase of the IFN response gene activity in the responders compared to the non-responders after three months of therapy (p=0.0317, FIG. 3).

Subsequently, we also compared differences between baseline values of several clinical parameters indicated between the two groups. This analysis revealed no associations between the differential regulation of IFN type I response activity and the clinical parameters presented in Table 1 (data not shown).

Thus, whereas rituximab depletes B-cells in all patients treated, irrespective of their clinical response, our data show that pharmacodynamic differences in the type I IFN response activity discriminates between clinical good responders and non-responders to rituximab treatment based on both DAS28 and EULAR criteria.

In summary, these findings mark a pharmacological mechanism of action that relies on the induction of changes in the type I IFN response gene activity, which mark concomitant presence of agonistic IFN proteins and/or IFN-inducing agents, upon B-cell depletion by rituximab. B-cells that are targeted via anti-CD20, e.g., rituximab (an anti-CD20 antibody), are rapidly depleted from the peripheral blood CD20-positive B-cells via complement-mediated and antibody-dependent cell-mediated cytotoxicity (ADCC), induction of apoptosis and inhibition of cell growth (D. G. Maloney et al., Rituximab: Mechanism of action and resistance, Semin. Oncol. 2002, 29:2-9). The subsequent release from apoptotic/necrotic material from depleted cells is likely to promote IFN production and release, via TLR-mediated cell activation (S. Akira, S. Uematsu, O. Takeuchi, 2006, Pathogen recognition and innate immunity, Cell 124:783-801). The released IFN, on its turn, is then responsible for the relatively high increase in IFN type I response activity in patients who exhibit relatively low IFN type I response activity prior to therapy.

These results assigned the IFN pathway as an important pathway that determines the responder status of targeted B-cell depletion therapy. Knowing the divergent effects of IFN in disease pathogenesis, the concomitant activation of type I IFN may not always be beneficial. The IFN induction in RA is shown to be associated with a beneficial response. A similar mechanism will apply for a disease like MS, where IFNb is known to be beneficial in a subset of the patients. However, the concomitant release of type I IFN bioactivity, IFN type I-like bioactivity and/or type I IFN response activity may have detrimental effects in diseases such as SLE, wherein type I IFN contributes to disease pathogenesis, disease activity and/or disease severity.

### Association Between Type I IFN Pathway Activity and B-Cell Characteristics

The differences in type I IFN pathway activity were related to B-cell characteristics at baseline and during treatment in order to determine the possible role of IFN activity in treatment response. No significant correlation was found between baseline gene expression levels of CD 19 as a marker for B-cell count and baseline type I IFN pathway activity nor treatment-induced activity. However, a significant positive correlation was observed between MxA (vergelijking nog even doen met hele IFN cluster) and BAFF (B-cell-activating factor) at baseline (p=0.0145, r=0.6822) and after three months (p=0.0017, r=0.8013). Furthermore, a trend toward a significant positive correlation was observed between BAFF induction and baseline CD19 levels (p=0.0653, r=0.5477). Interestingly, all patients with low CD19 baseline levels show a decrease of BAFF expression after treatment in contrast to the increase that has been described in literature so far.

### Genetics and IFN Response Signature

In multiple sclerosis, we determined the association of three SNPs and the 30 by insertion-deletion polymorphism in the IRF5 gene with IFN type I response gene activity at baseline and after pharmacological intervention with IFN-beta. For rs2004640, we showed that patients homozygous for the T-allele have a significant higher baseline IFN type I response gene expression (P=0.0198) than heterozygous patients. Accordingly, a significantly reduced biological response was observed for patients homozygous for the T-allele versus heterozygous patients (P=0.0057) and patients homozygous for the G-allele (0.0340). For rs4728142, patients homozygous for the A-allele have a significantly higher baseline IFN type I response gene expression (P=0.0394) than heterozygous patients and a trend toward a lower biological response than heterozygous patients (p=0.1198) and homozygous for the G-allele (p=0.1421).

We claim the use of rs2004640 and rs4728142 as a marker for IFN activity in RA and for the use of predicting and monitoring therapy response with biologicals. Rs2004640 TT and rs4728142 AA patients are anticipated to have a high baseline IFN level and, thus, correspond with a bad response to BCIDT and/or TCIDT.

Tables 1.

TABLE 1A
Genes whose expression at baseline correlated with clinical
response (all genes correlation r = 0.6418, selected genes r = 0.8377)

| | correlation | correlation |
|---|---|---|
| | 0.6418) | 0.8377 |
| Homo sapiens interferon-induced transmembrane protein 1 (9-27) | X | |
| (IFITM1), mRNA. | | |
| Homo sapiens spleen focus forming virus (SFFV) proviral integration | X | |
| oncogene spi1 (SPI1), transcript variant 2, mRNA. | | |
| Homo sapiens flotillin 1 (FLOT1), mRNA. | X | |
| Homo sapiens ATH1, acid trehalase-like 1 (yeast) (ATHL1), mRNA. | X | |
| Homo sapiens myosin IF (MYO1F), mRNA. | X | |
| Homo sapiens ring finger protein 24 (RNF24), mRNA. | X | |
| Homo sapiens colony stimulating factor 3 receptor (granulocyte) | X | |
| (CSF3R), transcript variant 1, mRNA. | | |
| wi20e09.x1 NCI_CGAP_Co16 Homo sapiens cDNA clone | X | |
| IMAGE: 2390824 3, mRNA sequence | | |
| PREDICTED: Homo sapiens ankyrin repeat domain 13 family, | X | |
| member D, transcript variant 7 (ANKRD13D), mRNA. | | |
| Homo sapiens disrupted in schizophrenia 1 (DISC1), transcript variant | X | |
| S, mRNA. | | |
| Homo sapiens eukaryotic translation initiation factor 2-alpha kinase 2 | X | |
| (EIF2AK2), mRNA. | | |
| Homo sapiens 2′,5′-oligoadenylate synthetase 1, 40/46 kDa (OAS1), | X | |
| transcript variant 2, mRNA. | | |
| Homo sapiens metallothionein 2A (MT2A), mRNA. | X | |
| Homo sapiens metallothionein 1A (MT1A), mRNA. | X | |
| Homo sapiens interferon, alpha-inducible protein 27 (IFI27), mRNA. | X | |
| Homo sapiens peroxisomal proliferator-activated receptor A | X | X |
| interacting complex 285 (PRIC285), transcript variant 2, mRNA. | | |
| Homo sapiens interferon-induced transmembrane protein 3 (1-8U) | X | X |
| (IFITM3), mRNA. | | |
| Homo sapiens myxovirus (influenza virus) resistance 1, | X | X |
| interferon-inducible protein p78 (mouse) (MX1), mRNA. | | |
| Homo sapiens myxovirus (influenza virus) resistance 2 (mouse) | X | X |
| (MX2), mRNA. | | |
| Homo sapiens ISG15 ubiquitin-like modifier (ISG15), mRNA. | X | X |
| Homo sapiens poly (ADP-ribose) polymerase family, member 14 | X | X |
| (PARP14), mRNA. | | |
| Homo sapiens poly (ADP-ribose) polymerase family, member 12 | X | X |
| (PARP12), mRNA. | | |
| Homo sapiens lymphocyte antigen 6 complex, locus E (LY6E), | X | X |
| mRNA. | | |
| Homo sapiens XIAP associated factor 1 (XAF1), transcript variant 2, | X | X |
| mRNA. | | |
| Homo sapiens 2′-5′-oligoadenylate synthetase 3, 100 kDa (OAS3), | X | X |
| mRNA. | | |
| Homo sapiens radical S-adenosyl methionine domain containing 2 | X | X |
| (RSAD2), mRNA. | | |
| Homo sapiens interferon-induced protein 44-like (IFI44L), mRNA. | X | X |
| Homo sapiens hect domain and RLD 5 (HERC5), mRNA. | X | X |
| Homo sapiens interferon-induced protein 44 (IFI44), mRNA. | X | X |
| Homo sapiens epithelial stromal interaction 1 (breast) (EPSTI1), | X | X |
| transcript variant 2, mRNA. | | |
| Homo sapiens tripartite motif-containing 22 (TRIM22), mRNA. | X | X |
| Homo sapiens interferon-induced protein with tetratricopeptide repeats | X | X |
| 2 (IFIT2), mRNA. | | |
| Homo sapiens 2′,5′-oligoadenylate synthetase 1, 40/46 kDa (OAS1), | X | X |
| transcript variant 3, mRNA. | | |
| Homo sapiens 2′-5′-oligoadenylate synthetase-like (OASL), transcript | X | X |
| variant 2, mRNA. | | |
| Homo sapiens interferon-induced protein with tetratricopeptide repeats | X | X |
| 3 (IFIT3), mRNA. | | |
| Homo sapiens 2′-5′-oligoadenylate synthetase-like (OASL), transcript | X | X |
| variant 1, mRNA. | | |
| Homo sapiens interferon, alpha-inducible protein 6 (IFI6), transcript | X | X |
| variant 2, mRNA. | | |
| Homo sapiens toll-like receptor 5 (TLR5), mRNA. | X | |
| Homo sapiens hypothetical protein LOC153561 (LOC153561), | X | |
| mRNA. | | |
| Homo sapiens hairy and enhancer of split 4 (Drosophila) (HES4), | X | |
| mRNA. | | |

TABLE 1B
Genes whose expression at baseline correlated with clinical
response, rated on decreasing fold-difference (<0.7)

| Gene Name | Fold Change |
|---|---|
| IFI44L | 0.166355881 |
| LY6E | 0.200047979 |
| HERC5 | 0.219881515 |
| MX1 | 0.258197325 |
| IFITM3 | 0.307807163 |
| ISG15 | 0.32632231 |
| RSAD2 | 0.328904509 |
| IFI44 | 0.332874976 |
| EPSTI1 | 0.347643012 |
| IFI27 | 0.406435583 |
| HLA-A29.1 | 0.430776913 |
| IFIT3 | 0.46190674 |
| MX2 | 0.465392664 |
| PARP12 | 0.482702435 |
| IFIT2 | 0.488928415 |
| IFIT1 | 0.492501642 |
| MT2A | 0.501691656 |
| OASL | 0.523744525 |
| IFI6 | 0.52553565 |
| HLA-DRB5 | 0.53357054 |
| XAF1 | 0.535434784 |
| SHISA5 | 0.555303959 |
| IFITM1 | 0.562722366 |
| OAS3 | 0.56654893 |
| RNF24 | 0.56658262 |
| RNASE6 | 0.567300967 |
| PRIC285 | 0.573752976 |
| IFI35 | 0.576267343 |
| HES4 | 0.585124581 |
| DHX58 | 0.588144809 |
| OAS1 | 0.590981894 |
| TRIM22 | 0.592527473 |
| EIF2AK2 | 0.59293593 |
| MT1A | 0.593688112 |
| HERC6 | 0.599749038 |
| LOC642113 | 0.60565871 |
| UNC93B1 | 0.606923044 |
| PARP14 | 0.607148187 |
| PGS1 | 0.611341047 |
| NOD2 | 0.61578647 |
| MXD3 | 0.622961487 |
| OAS2 | 0.63323233 |
| HLA-DRB6 | 0.634533297 |
| CST7 | 0.637740886 |
| NRGN | 0.638377805 |
| SAMD9L | 0.639038086 |
| PSCD4 | 0.639293522 |
| ZBP1 | 0.640763582 |
| DAAM2 | 0.640898961 |
| DRAP1 | 0.641305266 |
| SCO2 | 0.647482264 |
| AXUD1 | 0.647813592 |
| SHKBP1 | 0.649862795 |
| JUNB | 0.651382168 |
| ATG16L2 | 0.661673659 |
| STAT2 | 0.66738189 |
| VWF | 0.676393645 |
| NKG7 | 0.677197496 |
| CCDC23 | 0.679387569 |
| SNHG5 | 0.680453694 |
| COL9A2 | 0.680881127 |
| MTHFR | 0.681805694 |
| FAM129A | 0.682221807 |
| HLA-H | 0.682579013 |
| GZMH | 0.683846883 |
| ATHL1 | 0.684490623 |
| REC8 | 0.684860582 |
| CCR1 | 0.68736306 |
| LOC441019 | 0.689004685 |
| RNF19B | 0.691046888 |
| RNF31 | 0.692610518 |
| MOV10 | 0.693790381 |
| FHL3 | 0.694464288 |
| MGC29506 | 0.694847468 |
| MYO1F | 0.695909149 |
| FLOT1 | 0.697032238 |
| HEATR1 | 0.697232166 |
| LOC127295 | 0.699127931 |

TABLE 1C
Genes whose expression at baseline correlated with DAS28
clinical response, rated on increasing q-value (False discovery Rate) from low to high.
Negative genes (12477)

| Row | Gene ID | Gene Name | Score (d) | Numerator ( [symbol] | Denominator (s + s0) | Fold Chang [symbol] | q |
|---|---|---|---|---|---|---|---|
| ### | Homo sa [symbol] | MX1 | −4.6975 | −1.82271783 | 0.388017975 | 0.25819733 | |
| ### | Homo sa [symbol] | SLC39A1 | −4.2454 | −0.44766371 | 0.1054461771 | 0.73209284 | |
| ### | Homo sa [symbol] | PARP12 | −4.0456 | −0.96523918 | 0.238591955 | 0.48270244 | |
| ### | Homo sa [symbol] | FAM46A | −3.996 | −0.39267766 | 0.098266835 | 0.75872853 | |
| ### | Homo sa [symbol] | CDCA3 | −3.873 | −0.16733152 | 0.043204261 | 0.89055913 | |
| ### | Homo sa [symbol] | ISG15 | −3.8705 | −1.38614628 | 0.358133408 | 0.32632231 | |
| ### | Homo sa [symbol] | ROPN1 | −3.6612 | −0.15112012 | 0.041276169 | 0.90092235 | |
| 828 | Homo sa [symbol] | APRIN | −3.6376 | −0.13385526 | 0.03679765 | 0.91149505 | |
| ### | Homo sa [symbol] | IFI35 | −3.5776 | −0.77400992 | 0.216348537 | 0.57626734 | |
| ### | Homo sa [symbol] | PERLD1 | −3.5633 | −0.3398056 | 0.095363415 | 0.79017767 | |
| ### | Homo sa [symbol] | IFI6 | −3.4651 | −0.84381226 | 0.243517249 | 0.52553565 | |
| ### | Homo sa [symbol] | C3orf45 | −3.413 | −0.15690841 | 0.045973957 | 0.89593057 | |
| ### | Homo sa [symbol] | DRAP1 | −3.3605 | −0.61539166 | 0.183127065 | 0.64130527 | |
| ### | Homo sa [symbol] | HERC5 | −3.3161 | −1.70094134 | 0.512928623 | 0.21988152 | |
| ### | Homo sa [symbol] | UNC93B1 | −3.2611 | −0.67292986 | 0.206351117 | 0.60692304 | |
| ### | Homo sa [symbol] | DNAL4 | −3.2397 | −0.22124649 | 0.068292385 | 0.85776225 | |
| ### | Homo sa [symbol] | VWF | −3.2374 | −0.5509846 | 0.170194075 | 0.67639364 | |
| ### | Homo sa [symbol] | OR52E8 | −3.2312 | −0.16221585 | 0.050202584 | 0.892805 | |
| ### | Homo sa [symbol] | DHX58 | −3.2028 | −0.6871021 | 0.214533853 | 0.58814481 | |
| ### | Homo sa [symbol] | LY6E | −3.1774 | −1.75159868 | 0.551259429 | 0.20004798 | |
| ### | Homo sa [symbol] | PSCD4 | −3.1563 | −0.61366066 | 0.194423871 | 0.63929352 | |
| ### | Homo sa [symbol] | IFI44L | −3.1512 | −1.87468602 | 0.594906225 | 0.16635588 | |
| ### | Homo sa [symbol] | GLIS2 | −3.1456 | −0.11366149 | 0.036133996 | 0.92388785 | |
| ### | Homo sa [symbol] | MICB | −3.1147 | −0.24775904 | 0.079544977 | 0.84568612 | |
| ### | Homo sa [symbol] | SLC22A18 | −3.0992 | −0.3697264 | 0.119296479 | 0.76484348 | |
| ### | Homo sa [symbol] | REC8 | −3.0966 | −0.50393102 | 0.162738393 | 0.68486058 | |
| ### | Homo sa [symbol] | SHISA5 | −3.0847 | −0.74249111 | 0.240700538 | 0.55530396 | |
| ### | Homo sa [symbol] | IFIT1 | −3.0637 | −0.87499254 | 0.285040268 | 0.49250164 | |
| ### | Homo sa [symbol] | ZHX3 | −3.0423 | −0.23518412 | 0.077304607 | 0.84847007 | |
| ### | Homo sa [symbol] | TAAR2 | −3.0414 | −0.19018096 | 0.062530376 | 0.8738634 | |
| ### | Homo sa [symbol] | C1QB | −3.0386 | −0.39745757 | 0.130801454 | 0.75956256 | |
| ### | Homo sa [symbol] | SP140 | −3.0341 | −0.28941029 | 0.095385657 | 0.81782871 | |

[symbol]

indicates data missing or illegible when filed

TABLE 1D
Genes whose expression at baseline correlated with EULAR
clinical response, rated on increasing q-value (False discovery Rate) from low to high
Negative genes (12420)

| Row | Gene ID | Gene Name | Score (d) | Numerator ( [symbol] | Denominator (s + s0) | Fold Chang [symbol] | q |
|---|---|---|---|---|---|---|---|
| ### | Homo sa [symbol] | MX1 | −6.1654 | −2.00766685 | 0.32563637 | 0.24029237 | |
| ### | | FAM46A | −5.3351 | −0.43801593 | 0.082100555 | 0.73762832 | |
| ### | Homo sa [symbol] | IFI6 | −4.9533 | −0.98759164 | 0.199379682 | 0.48502565 | |
| ### | Homo sa [symbol] | ENDOGL1 | −4.8552 | −0.5112036 | 0.105288981 | 0.69381897 | |
| ### | Homo sa [symbol] | ISG15 | −4.5468 | −1.50668624 | 0.331369405 | 0.30767973 | |
| ### | Homo sa [symbol] | LOC647625 | −4.4746 | −0.14597546 | 0.032623247 | 0.90357645 | |
| ### | Homo sa [symbol] | HERC5 | −4.3101 | −1.94795133 | 0.451954307 | 0.1962335 | |
| ### | Homo sa [symbol] | LDB1 | −4.289 | −0.1989191 | 0.046373744 | 0.87203402 | |
| ### | Homo sa [symbol] | IFI44L | −4.193 | −2.18144028 | 0.520262024 | 0.14559675 | |
| ### | Homo sa [symbol] | IFIT1 | −4.1217 | −1.02242413 | 0.248059141 | 0.4522264 | |
| ### | Homo sa [symbol] | PLEKHM3 | −4.1019 | −0.14725518 | 0.03589937 | 0.9029998 | |
| ### | Homo sa [symbol] | PRIC285 | −4.0879 | −0.89739153 | 0.219526117 | 0.52553882 | |
| ### | Homo sa [symbol] | SHISA5 | −4.0864 | −0.86162284 | 0.210851671 | 0.51702962 | |
| ### | Homo sa [symbol] | DNAL4 | −4.0753 | −0.24648311 | 0.06048229 | 0.8436481 | |
| ### | Homo sa [symbol] | IRFZ | −4.0688 | −0.47695428 | 0.117221322 | 0.70772861 | |
| ### | Homo sa [symbol] | PARP12 | −3.9622 | −0.97835889 | 0.246923753 | 0.48161907 | |
| ### | Homo sa [symbol] | TRIM38 | −3.9522 | −0.46614315 | 0.117944669 | 0.72232255 | |
| ### | Homo sa [symbol] | LY6E | −3.9476 | −1.98139955 | 0.501926109 | 0.17923892 | |
| ### | Homo sa [symbol] | VWF | −3.8407 | −0.60725227 | 0.158109507 | 0.6527704 | |
| ### | Homo sa [symbol] | MX2 | −3.8341 | −1.16524784 | 0.303914936 | 0.41709754 | |
| ### | Homo sa [symbol] | XAF1 | −3.7265 | −0.94385793 | 0.25328038 | 0.48298912 | |
| ### | Homo sa [symbol] | SCGB1C1 | −3.6938 | −0.34168543 | 0.092501515 | 0.78355838 | |
| ### | Homo sa [symbol] | REC8 | −3.6926 | −0.55842049 | 0.151226613 | 0.66127907 | |
| ### | Homo sa [symbol] | CDCA3 | −3.6705 | −0.16584872 | 0.045183879 | 0.89174382 | |
| ### | Homo sa [symbol] | DHX58 | −3.6704 | −0.7479208 | 0.203769311 | 0.56564404 | |
| ### | Homo sa [symbol] | DRAP1 | −3.6451 | −0.65180931 | 0.17882 | 0.62679486 | |
| ### | Homo sa [symbol] | IL17A | −3.566 | −0.15244087 | 0.42748362 | 0.89973967 | |
| ### | Homo sa [symbol] | N4BP1 | −3.5515 | −0.26162486 | 0.073665106 | 0.83226342 | |
| 632 | Homo sa [symbol] | ANKFY1 | −3.5442 | −0.31691827 | 0.089419823 | 0.80127531 | |
| ### | Homo sa [symbol] | UNC93B1 | −3.5422 | −0.71441122 | 0.201686145 | 0.59092213 | |
| ### | Homo sa [symbol] | RSAD2 | −3.5387 | −1.43585003 | 0.405755648 | 0.28997959 | |
| ### | PREDIC [symbol] | LOC647046 | −3.5348 | −0.18118739 | 0.051258352 | 0.87993774 | |

[symbol]

indicates data missing or illegible when filed

TABLE 2
Genes whose expression changed from baseline (T0) until three
months after therapy (T3) correlated with clinical response
(all genes correlation r = 0.5336, selected genes r = 0.7857)

| | node 377x | node 352x |
|---|---|---|
| | correlation | correlation |
| Ratio T3/T0 | 0.5336 | 0.7857 |
| Homo sapiens interferon, alpha-inducible protein 27 (IFI27), | X | |
| mRNA. | | |
| Homo sapiens 2′,5′-oligoadenylate synthetase 1, 40/46 kDa | X | |
| (OAS1), transcript variant 2, mRNA. | | |
| Homo sapiens interferon-induced protein with tetratricopeptide | X | |
| repeats 2 (IFIT2), mRNA. | | |
| Homo sapiens epithelial stromal interaction 1 (breast) | X | X |
| (EPSTI1), transcript variant 2, mRNA. | | |
| Homo sapiens interferon-induced protein 44-like (IFI44L), | X | X |
| mRNA. | | |
| Homo sapiens interferon-induced protein 44 (IFI44), mRNA. | X | X |
| Homo sapiens myxovirus (influenza virus) resistance 1, | X | X |
| interferon-inducible protein p78 (mouse) (MX1), mRNA. | | |
| Homo sapiens ISG15 ubiquitin-like modifier (ISG15), mRNA. | X | X |
| Homo sapiens lymphocyte antigen 6 complex, locus E (LY6E), | X | X |
| mRNA. | | |
| Homo sapiens interferon-induced transmembrane protein 3 | X | X |
| (1-8U) (IFITM3), mRNA. | | |
| Homo sapiens radical S-adenosyl methionine domain | X | X |
| containing 2 (RSAD2), mRNA. | | |
| Homo sapiens hect domain and RLD 5 (HERC5), mRNA. | X | X |
| Homo sapiens interferon, alpha-inducible protein 6 (IFI6), | X | X |
| transcript variant 3, mRNA. | | |
| Homo sapiens hairy and enhancer of split 4 (Drosophila) | X | X |
| (HES4), mRNA. | | |
| Homo sapiens interferon-induced protein with tetratricopeptide | X | X |
| repeats 3 (IFIT3), mRNA. | | |
| Homo sapiens interferon, alpha-inducible protein 6 (IFI6), | X | |
| transcript variant 2, mRNA. | | |
| Homo sapiens lysozyme (renal amyloidosis) (LYZ), mRNA. | X | |

1. A method for prognosticating the clinical response to a B-lymphocyte inhibiting or depleting agent (BCID) or a T-cell inhibiting or depleting agent (TCID) in a patient afflicted with a disease wherein IFN contributes to said disease, disease activity and/or severity, said method comprising:
providing a sample from the patient,
determining the level of an IFN response in said sample, and
prognosticating said clinical response from said IFN response,
wherein a low level of IFN response indicates the likelihood of a poor clinical response to said BCID or TCID.

2. The method according to claim 1, further comprising comparing the determined level of an IFN response in said sample to a reference.

3. The method according to claim 2, wherein said reference is selected from the group consisting of a) a reference value, b) the level of an IFN response in a second sample from the patient that has been exposed to a BCID or TCID, and c) the level of an IFN response in a second sample from the patient that has been exposed to an IFN or IFN-inducing agent.

4. A method for prognosticating the clinical response to an IFN or IFN-inducing agent in a patient afflicted with a disease, wherein IFN contributes to said disease, disease activity and/or severity and, said method comprising:
providing a sample from the patient,
determining the level of an IFN response in said sample, and
prognosticating said clinical response from said IFN response,
wherein a low level of IFN response indicates the likelihood of a poor clinical response to said IFN or IFN-inducing agent.

5. The method according to claim 4, further comprising comparing the determined level of an IFN response in said sample to a reference.

6. The method according to claim 5, wherein said reference is selected from the group consisting of a) a reference value, b) the level of an IFN response in a second sample from the patient that has been exposed to a B-lymphocyte inhibiting or depleting agent (BCID), and a T-cell inhibiting or depleting agent (TCID).

7. The method according to claim 3, wherein said reference value is obtained from one or more individuals not afflicted with a disease wherein IFN contributes to said disease, disease activity and/or severity.

8-12. (canceled)

13. The method of claim 10, wherein increased expression at baseline of said sample is associated with a good clinical response.

14. The method according to claim 1, wherein said IFN response level is determined by determining the expression level of BAFF and DARC genes supplemented with at least one gene selected from the group consisting of genes from Tables 1A, 1B, 1C, 1D and 2.

15. The method according to claim 1, wherein the IFN response level is determined by determining the level of an expression product of at least one gene selected from the group consisting of Mx1 (MxA), ISG15, OAS1, LGALS3BP, RSAD2, IFI44L, IFI44, Mx2 (MxB), OAS2, DARC, BAFF, HERC5, Ly6E, IFI27, RAP1GAP, EPSTI1 and/or SERPING1.

16. The method according to claim 1, wherein the IFN response level is determined by determining the level of an expression product of at least one gene selected from the group consisting of OAS 1 and Mx2

17. The method according to claim 1, wherein the IFN response level is determined by determining the level of an expression product of at least one gene selected from the group consisting of RSAD2 and IFI44L.

18. The method according to claim 1, wherein the IFN response level is determined by determining the level of an expression product of at least one gene selected from the group consisting of Mx1, ISG15, OAS2 and SERPING1.

19. The method according to claim 1, wherein said IFN response level is determined by determining the level of an expression product of a gene selected from the group consisting of genes listed in Tables 1A, 1B, 1C, 1D, Table 2, BAFF and DARC.

20. The method according to claim 1, wherein said sample comprises cells and serum/plasma.

21. The method according to claim 1, wherein said sample comprises cells and serum/plasma from the patient before the start of the therapy to predict the response to a soluble BCID or TCID agent.

22. The method according to claim 1, wherein said at least a second sample is obtained from an individual between 1 and 8 months after the first exposure of the patient to said a soluble BCID or TCID agent.

23. The method according to claim 1, wherein said at least a second sample also taken at baseline, has been exposed in vitro to said soluble BCID or TCID agent.

24. The method according to claim 1, wherein said at least a second sample also taken at baseline, has been exposed in vitro to IFN or an IFN-inducing agent.

25. The method according to claim 1, wherein said at least a second sample has been obtained from a patient that has been exposed to a BCID or TCID agent.

26. The method
according to claim 1, further comprising:
treating the patient with a soluble BCID or TCID agent, if the patient has been prognosticated as a good responder.

27. The method according to claim 1, wherein said BCID is rituximab.

28. The method according to claim 1, wherein said disease is selected from the group consisting of systemic lupus erythematosus, Sjögren's disease, myositis, dermatomyositis, polymyositis and systemic sclerosis.

29. The method according to claim 28, wherein said disease is selected from the group consisting of systemic lupus erythematosus, Sjögren's disease, polymyositis and systemic sclerosis.

30. The method according to claim 1, wherein the patient has not previously been exposed to a BCID or TCID agent.

31. The method according to claim 30, wherein the patient has also not been exposed to an IFN or IFN-inducing agent.

32. The method according to claim 4, wherein the patient has not previously been exposed to a B-lymphocyte inhibiting or depleting agent (BCID) or a T-cell inhibiting or depleting agent (TCID).

33. The method according to claim 32, wherein the patient is a candidate for treatment with BCID or TCID.

34. The method according to claim 23, wherein said at least a second sample also taken at baseline, simultaneously with sample one prior to the start of therapy, has been exposed in vitro to a soluble BCID or TCID agent.

35. The method according to claim 24, wherein said at least a second sample also taken at baseline, simultaneously with sample one prior to the start of therapy, has been exposed in vitro to IFN or an IFN-inducing agent.

36. The method according to claim 35, wherein the IFN-inducing agent is dsDNA or dsRNA.

37. A method of treating a subject diagnosed as suffering from or at risk of suffering from systemic lupus erythematosus, Sjögren's disease, myositis, dermatomyositis, polymyositis, or systemic sclerosis, the method comprising:
determining the level of an IFN response in a sample from the subject;
prognosticating the clinical response of the subject to a soluble B-lymphocyte inhibiting or depleting agent (BCID) or a T-cell inhibiting or depleting agent (TCID) from the IFN response, wherein a low level of IFN response indicates the likelihood of a poor clinical response to a BCID or a TCID; and,
if the subject has not been prognosticated as likely having a poor clinical response, treating the subject with a soluble BCID or TCID.

38. The method according to claim 37, wherein the subject has not previously been exposed to a BCID or a TCID.

39. The method according to claim 38, wherein the subject has also not been exposed to an IFN or IFN-inducing agent.
